(19) United States
(12) Patent Application Publication
(10) Pub. No.: US 2025/0381110 A1
(43) Pub. Date: Dec. 18, 2025

(54) SKIN CARE COMPOSITION AND METHOD OF USING THE SAME

(71) Applicant: THE PROCTER & GAMBLE COMPANY, Cincinnati, OH (US)

(72) Inventors: Michael Joseph FLAGLER, Cincinnati, OH (US); Viktor Igorovich KOROLCHUK, Newcastle upon Tyne (GB); Charles Carson BASCOM, Fairfield Township, OH (US); Matthew Clair EHRMAN, Singapore (SG); Max Adam MUSHENS-BROWN, Newcastle upon Tyne (GB); John Erich OBLONG, Loveland, OH (US); Hilda Andamiche NAMANJA-MAGLIANO, Loveland, OH (US); Shikhar Gupta, Singapore (SG); Jason Kenneth Allen, Milford, OH (US)

(21) Appl. No.: 19/240,364

(22) Filed: Jun. 17, 2025

Related U.S. Application Data
(60) Provisional application No. 63/660,818, filed on Jun. 17, 2024.

(51) Int. Cl. A61K 8/365; A61K 8/34; A61K 8/37; A61Q 19/08

(52) U.S. Cl. CPC A61K 8/365; A61K 8/347; A61K 8/375; A61Q 19/08; A61K 2800/74; A61K 2800/805

(57) ABSTRACT

A skin care composition that includes bioactive materials, other optional skin ingredients. and a dermatologically acceptable carrier. The materials stimulate mitophagy and prevent or reverse cellular aging to help improve skin health and appearance.

### FIELD

The present disclosure is directed generally to improving health of the skin and mucosal epithelium with specific actives or bioactive materials. More specifically, the present disclosure is directed to materials which prevent and reverse cellular aging phenotypes by stimulating mitophagy.

### BACKGROUND

Consumers are very concerned about the impact of stress on their health. They recognize that the impact of acute stress (e.g., irritation, UV exposure) can accumulate and lead to longer term concerns across body sites, including manifestations such as skin wrinkles, gum recession, vaginal atrophy, and/or gastrointestinal issues. Therefore, younger consumers are now looking to combat the effects of stress earlier. Thus, approaches to intervene early in the cellular events that are activated by acute stress (e.g., oxidative stress, mitochondrial dysfunction) are necessary to reduce or prevent longer-term consumer noticeable concerns.

Skin is made up of a variety of different cells that function together in a dynamic, complex relationship to maintain the health of the tissue. However, as skin cells age or become damaged, they can lose their ability to function at the level needed to maintain young, healthy-looking skin. Skin cells can be damaged by a variety of endogenous and exogenous stressors (e.g., ultraviolet radiation, pollution, smoking). In some instances, these stressors can cause the production of reactive oxygen species (ROS), which interfere with normal cellular processes. In response, cells have evolved defenses to combat ROS, but the cell's defenses can be overwhelmed by spikes of stressor-induced ROS, leading to not just acute but also chronic alterations in cellular homeostasis. As ROS accumulate over time, they cause oxidative stress at the cellular level, which can ultimately manifest as visible signs of aging (e.g., fine lines, wrinkles, hyperpigmented spots, thinning skin).

Accordingly, it would be desirable to provide a skin care composition that can combat the effects of ROS and cellular events that are activated by acute stress (e.g., oxidative stress, mitochondrial dysfunction) to prevent and/or reverse longer-term consumer-noticeable concerns associated with a decline in skin health, especially skin that exhibits a visible sign of aging. In particular, it would be desirable to provide a skin care composition containing specific materials that improve key quality control processes in a skin cell that are impaired by oxidative stress and aging by targeting specific molecules involved in these biochemical pathways.

### SUMMARY

Disclosed herein is a skin care composition, comprising: a bioactive material; and a dermatologically acceptable carrier; wherein the composition increases mitophagy in human skin cells. Also disclosed is a method of treating a skin condition comprising applying the novel composition herein to a target portion of skin where treatment is desired.

### BRIEF DESCRIPTION OF THE DRAWINGS

While the specification concludes with claims particularly pointing out and distinctly claiming the subject matter that is regarded as the present disclosure, it is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 shows the chemical structure of STOCKIN-57543, a reference compound.

FIGS. 2A and 2B show fluorescence microscopy images and quantifications of primary human dermal fibroblasts (HDFs) isolated from skin biopsies from young or aged donors and expressing mt-mKeima. Cells were treated with STOCKIN-57543 (30 μM) for 7 h.

FIGS. 3A and 3B show immunostaining for p21 and quantifications of the percentage of cells positive for p21 and the average nuclear size in fibroblasts isolated from skin biopsies from young or aged donors. Cells were treated with STOCKIN-57543 (30 μM) for 7 h.

FIG. 4 shows EdU incorporation assay result in fibroblasts isolated from skin biopsies from young or aged donors (HDFs). Cells were treated with STOCKIN-57543 (30 μM) for 7 h.

FIG. 5 shows mitochondrial mass assessed by Mitotracker Green staining in fibroblasts isolated from skin biopsies from young or aged donors. Cells were treated with STOCKIN-57543(30 μM) for 7 h.

FIG. 6 shows ATP production per cell normalized to MitoTracker density in fibroblasts isolated from skin biopsies from young or aged donors. Cells were treated with STOCKIN-57543 (30 μM) for 7 h.

FIGS. 7A and 7B show cell motility assay results in fibroblasts isolated from skin biopsies from young or aged donors. Cells were treated with STOCKIN-57543 (30 μM) for 7 h.

FIG. 8 shows mRNA levels of IL-6 in fibroblasts isolated from skin biopsies from young or aged donors. Cells were treated with STOCKIN-57543 (30 μM) for 7 h.

FIGS. 9A and 9B show fluorescence microscopy images and quantifications of mitophagy events in HDFs expressing mt-mKeima. Cells were treated with STOCKIN-57534 (30 μM) for 5 h prior to 20 Gy IR and imaged at the timepoints indicated.

FIGS. 10A and 10B show immunostaining for p21 and quantification of the percentage of cells positive for p21 and the average nuclear size in fibroblasts isolated from skin biopsies from young or aged donors. Cells were treated with STOCKIN-57534 (30 μM) for 7 h.

FIG. 11 shows mRNA levels of IL-6 and IL-8 in HDFs before and 11 days after IR. Cells were treated with STOCKIN-57534 (30 μM) for 5 h prior to IR.

FIGS. 12A and 12B show immunostaining for Ki67 and quantifications of the percentage of cells positive for Ki67 in HDFs before and 11 days after IR. Cells were treated with STOCKIN-57534 (30 μM) for 5 h prior to IR.

FIG. 13 shows in silico docking predictions of selected sterane compounds to the binding pocket of p62 ZZ domain (PDB ID: 6MJ7).

FIGS. 14A-14M show sterane structures and associated docking scores used to establish a Markush structure representing p62 ZZ domain-binding sterane compounds. p62-dependent mitophagy values are included for those compounds tested in the Luciferase-p62 assay.

FIGS. 15A-15C show hydroxycinnamic acids (HCAs) tested in mitophagy assays in primary HDFs. A) p-coumaric acid (pCA) (5 μM) significantly increases p62-dependent mitophagy in HDFs vs. untreated control cells in the Luciferase-p62 clearance assay. B) pCA (8 μM) increases mitophagy events as measured by fluorescence microscopy. 80,000 mixed aged Fibroblasts expressing the Su9-Halo-GFP reporter were treated for 48 h with the indicated HCAs. A change in the reporter excitation occurs because the GFP tag is quenched in the lysosome environment, indicating mitophagy.

FIG. 16 shows HDFs were treated with 400 uM H₂O₂ for 2 hours to introduce oxidative stress, then treated in triplicates followed by incubation for 22 h at 37° C., 5% CO₂ followed by S-β-Gal staining. Compound A, p-coumaric acid.

FIGS. 17A and 17B shows dose-response testing of two different HCA chemistries in the mitophagy assay described in FIG. 15. A monohydroxycinnamic acid (p-coumaric acid) outperforms an O-methylated form (ferulic acid) for increasing basal mitophagy levels in HDFs between the concentrations of 0.1-8 μM.

### DETAILED DESCRIPTION

The importance of selective autophagic clearance of mitochondria (the cellular process known as mitophagy) in skin cellular physiology has not previously been explored in detail, despite its predicted role in the maintenance of mitochondrial quality control and cellular function in other tissues. We have demonstrated herein that failure of mitophagy leads to cellular aging through enhanced cellular stress and senescence, supporting a strong dependence of skin cells on functional mitophagy. By focusing on human dermal fibroblasts (HDFs), we have delineated the triggers and the molecular machinery involved in maintaining basal mitophagy in human skin cells. Furthermore, we have discovered that mitophagy is downregulated in response to acute stress and chronological aging in skin cells, identifying this event as a driver of cellular aging phenotypes. In addition, we have demonstrated that cellular senescence and aging phenotypes can be reversed by bioactive materials which act to maintain or restore functional mitophagy in skin cells. Specifically, we identified materials that stimulate mitophagy and reverse cellular aging via modulation of p62 functionality in this process. Additionally, these materials can increase p62-dependent mitophagy levels in skin cells under stress conditions in which mitophagy is suppressed, including models of senescence induced by irradiation (IR), thereby preventing IR-induced cellular senescence and aging. It is possible that the benefits of these materials could be extended to other stress-induced triggers of cellular senescence and aging (e.g. inflammation, ROS-induced oxidative stress, DNA damage) and epithelial tissues (e.g., oral mucosal epithelium, vaginal epithelium). Taken together, our data indicate that mitophagy represents a promising target for the development of anti-aging strategies, and this critical quality control process can be enhanced by treatment of skin cells with bioactive materials which act upon specific cellular targets.

The use of materials which work via physical and biological mechanisms to improve the health and appearance of skin is generally known. However, it has now been surprisingly discovered that bioactive materials can maintain mitophagy levels in skin cells exposed to acute cellular stress as well as restore functional mitophagy in skin cells from aged individuals, in which this critical quality control process is impaired. Mitophagy is process which is part of a cell's metabolism and is increasingly recognized as a key cellular quality control mechanism, perturbation of which may contribute to the development of age-related diseases (Sedlackova et al.).

Moreover, we have demonstrated that mitophagy dysfunction is an important mechanism in the development of cellular senescence. Cellular senescence is triggered by irreparable damage, resulting in a permanent cell cycle arrest. The mechanisms leading to senescence acquisition are complex, and the key drivers that activate the senescence program remain unclear. Senescent cells are typically characterized by a set of markers such as persistent DNA damage, elevated levels of reactive oxygen species (ROS), increased cell size with an expansion of cellular organelles including the nucleus and lysosomal compartment (the latter visualized by the senescence-associated β-galactosidase [SA-β-GAL] staining), and senescence-associated secretory phenotype (SASP) (Korolchuk et al.). Cellular senescence has now emerged as an important element of organismal aging, which is associated with a gradual accumulation of senescent cells in various tissues that has been shown to contribute to the age-related functional decline (Gorgoulis et al.). We have identified specific materials which can increase mitophagy levels in skin cells and either prevent or rescue the cellular senescence and aging phenotype. Restoring functional mitophagy under stress and aging conditions which are encountered in people's everyday lives is important for improving the health and appearance of skin, especially skin that exhibits visible signs of aging.

Reference herein to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all ingredient percentages are based on the weight of the cosmetic composition, unless specifically stated otherwise. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive and combinable to form narrower ranges not explicitly disclosed. For example, delineated upper and lower range limits are interchangeable to create further ranges.

The compositions of the present invention can comprise, consist essentially of, or consist of, the essential components as well as optional ingredients described herein. As used herein, “consisting essentially of” means that the composition or component may only include additional ingredients that do not materially alter the basic and novel characteristics of the claimed composition or method. As used in the description and the appended claims, the singular forms “a”, “an”, and “the” are intended to include the plural forms as well, unless the context clearly indicates otherwise.

### Definitions

“About” modifies a particular value by referring to a range equal to plus or minus twenty percent (+/−20%) or less (e.g., less than 15%, 10%, or even less than 5%) of the stated value.

“Apply” or “application”, as used in reference to a composition, means to apply or spread the compositions of the present invention onto a human skin surface such as the epidermis.

“Bioactive material” or “bioactive compound” means a compound or combination of compounds that, when applied to skin, provide an acute and/or chronic benefit to skin or a type of cell commonly found therein. They may regulate and/or improve skin or its associated cells (e.g., improve skin elasticity, hydration, skin barrier function, and/or cell metabolism). Bioactive materials or bioactive compounds may be derived from nature, i.e., naturally derived, or chemically synthesized. Bioactive materials or bioactive compounds may be skin care actives.

“Cosmetic composition” means a composition comprising a cosmetic agent and intended for non-therapeutic (i.e., non-medical) use. Examples of cosmetic compositions include color cosmetics (e.g., foundations, lipsticks, concealers, and mascaras), skin care compositions (e.g., moisturizers and sunscreens), personal care compositions (e.g., rinse-off and leave on body washes and soaps), hair care compositions (e.g., shampoos and conditioners).

“Derivative,” herein, means amide, ether, ester, amino, carboxyl, acetyl, and/or alcohol derivatives of a given compound, or bioactive material.

“Effective amount” means an amount of a compound, bioactive material or composition sufficient to significantly induce a positive benefit to keratinous tissue over the course of a treatment period. The positive benefit may be a health, appearance, and/or feel benefit, including, independently or in combination, the benefits disclosed herein.

“Skin care” means regulating and/or improving a skin condition (e.g., skin health, appearance, or texture/feel). Some nonlimiting examples of improving a skin condition include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Treatment period,” as used herein, means the length of time and/or frequency that a material or composition is applied to a target skin surface.

### Skin Care Composition

The novel skin care compositions herein are intended for topical application to human skin to enhance or restore functional mitophagy to prevent and/or reverse the effects of mitochondrial dysfunction on cellular aging. The present skin care compositions contain a safe and effective amount of bioactive materials that stimulate mitophagy.

The skin care compositions herein may be cosmetic compositions, pharmaceutical compositions, or cosmeceutical compositions, and may be provided in various product forms, including, but not limited to, solutions, suspensions, lotions, creams, gels, toners, sticks, sprays, aerosols, ointments, cleansing liquid washes and solid bars, pastes, foams, mousses, shaving creams, wipes, strips, patches, electrically-powered patches, hydrogels, film-forming products, facial and skin masks (with and without insoluble sheet), make-up such as foundations, eye liners, and eye shadows, and the like. In some instances, the composition form may follow from the particular dermatologically acceptable carrier chosen. For example, the composition (and carrier) may be provided in the form of an emulsion (e.g., water-in-oil, oil-in-water, or water-in-oil-in water) or an aqueous dispersion.

The compositions herein may be prepared by conventional methods of making topical skin care compositions. Such methods typically involve mixing of the ingredients in one or more steps to a relatively uniform state, with or without heating, cooling, application of vacuum, and the like. The compositions are preferably prepared such as to optimize stability (physical stability, chemical stability, photostability) and/or delivery of the bioactive materials. This optimization may include appropriate pH (e.g., less than 7), exclusion of materials that can complex with the bioactive material and thus negatively impact stability or delivery (e.g., exclusion of contaminating iron), use of approaches to prevent complex formation (e.g., appropriate dispersing agents or dual compartment packaging), use of appropriate photostability approaches (e.g., incorporation of sunscreen/sunblock, use of opaque packaging), etc.

### Vitamin B₃ Compound

The compositions herein may optionally include a safe and effective amount of a vitamin B₃ compound. In some instances, the present compositions may contain 0.01% to 10%, by weight, of the vitamin B₃ compound, based on the weight or volume of the composition (e.g., 0.1% to 10%, 0.5% to 5%, or even 1% to %).

As used herein, “vitamin B₃ compound” means a compound having the formula:

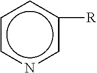

Where: R is CONH₂ (i.e., niacinamide), COOH (i.e., nicotinic acid) or CH₂OH (i.e., nicotinyl alcohol); derivatives thereof; and salts of any of the foregoing.

Exemplary derivatives of vitamin B₃ compounds include nicotinic acid esters, including non-vasodilating esters of nicotinic acid (e.g., tocopheryl nicotinate, myristyl nicotinate) nicotinamide riboside, nicotinyl amino acids, nicotinyl alcohol esters of carboxylic acids, nicotinic acid N-oxide, and niacinamide N-oxide.

The compositions herein include a safe and effective amount of one or more mitophagy stimulating bioactive materials in Table 2, or compounds having >90% similarity with the bioactive materials described in the Table 2 as determined by EPFC fingerprint descriptors and Tanimoto coefficient (Table 3), or a hydroxycinnamic acid. Hydroxycinnamic acids are an example of bioactive materials. In some instances, the bioactive materials or bioactive compounds may be present in the present compositions at 0.01-100 μM, for example, 0.01-0.1 μM, 0.01-0.5 μM, 0.01-1 μM, 1 M-2 μM, 1-5 μM, 5-10 μM, 10-25 μM, 25-50 μM, 50-75 μM, or 75-100 μM. In some instances, the bioactive materials or bioactive compounds may be present in the present compositions at 0.01%-10% by weight of the total composition, or 0.01%-0.1%, 0.01%-0.5%, 0.01%-1%, 0.1%-1%, 0.1%-2%, 0.1%-5%, 1%-5%, 1%-10%, or 5%-10%.

### Dermatologically Acceptable Carrier

The compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the bioactive materials in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In some instances, the dermatologically acceptable carrier is in the form of an emulsion that has a continuous aqueous phase (e.g., an oil-in-water or water-in-oil-in-water emulsion) or a continuous oil phase (e.g., water-in-oil or oil-in-water-in-oil emulsion). The oil phase of the emulsion may include silicone oils, non-silicone oils such as hydrocarbon oils, esters, ethers, and mixtures thereof. The aqueous phase may include water and water-soluble ingredients (e.g., water-soluble moisturizing agents, conditioning agents, anti-microbials, humectants and/or other skin care actives). In some instances, the aqueous phase may include components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s).

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable diluents. As used herein. “diluent” refers to materials in which the skin care actives herein can be dispersed, dissolved, or otherwise incorporated. Some non-limiting examples of hydrophilic diluents include water, organic hydrophilic diluents such as lower monovalent alcohols (e.g., C₁-C₄) and low molecular weight glycols and polyols, including propylene glycol, polyethylene glycol (e.g., molecular weight of 200 to 600 g/mole), polypropylene glycol (e.g., molecular weight of 425 to 2025 g/mole), glycerol, butylene glycol, 1,2,4-butanetriol, sorbitol esters, 1,2,6-bexanetriol, ethanol, isopropanol, butanediol, ether propanol, ethoxylated ethers, propoxylated ethers and combinations thereof.

### Conditioning Agents

The compositions herein may include 0.1% to 50% by weight of a conditioning agent (e.g., 0.5% to 30%, 1% to 20%, or even 2% to 15%). Adding a conditioning agent can help provide the composition with desirable feel properties (e.g., a silky, lubricious feel upon application). Some non-limiting examples of conditioning agents include, hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof. Particularly suitable examples of conditioning agents include volatile or non-volatile silicone fluids such as dimethicone copolyol, dimethylpolysiloxane, diethylpolysiloxane, mixed C1-30 alkyl polysiloxanes, phenyl dimethicone, dimethiconol, dimethicone, dimethiconol, silicone crosspolymers, and combinations thereof. Dimethicone may be especially suitable, since some consumers associate the feel properties provided by certain dimethicone fluids with good moisturization. Other examples of silicone fluids that may be suitable for use as conditioning agents are described in U.S. Pat. No. 5,011,681.

### Rheology Modifiers

The compositions herein may include 0.1% to 5% of a rheology modifier (e.g., thickening agent) to provide the composition with suitable rheological and skin feels properties. Some non- limiting examples of thickening agents include crosslinked polyacrylate polymers, polyacrylamide polymers, polysaccharides, gums and mixtures thereof. In a particularly suitable example, the composition may include a superabsorbent polymer thickening agent such as sodium polyacrylate, starch grafted sodium polyacrylate, or a combination of these. Some non-limiting examples of superabsorbent polymer thickeners are described in, for example, U.S. Pat. No. 9,795,552.

Some consumers find compositions that use silicone fluids as conditioning agents to be undesirably greasy or heavy feeling. Thus, it may be desirable to provide a composition that is free of or substantially free of silicone fluid. It may also be desirable to tailor a superabsorbent polymer thickener to provide the composition with a light, airy feel, for example, by adjusting the amount of water in the composition, the water: oil ratio (e.g., 12:1 to 1:1), and/or the ratio of water to thickener or oil to thickener.

### Emulsifiers

When the dermatologically acceptable carrier is in the form of an emulsion, it may be desirable to include an emulsifier to provide a stable composition (e.g., does not phase separate). When included, the emulsifier may be present at an amount of 0.1% to 10% (e.g., 1% to 5%, or 2%-4%). Emulsifiers may be nonionic, anionic or cationic. Some non-limiting examples of emulsifiers that may be suitable for use herein are disclosed in U.S. Pat. Nos. 3,755,560; 4,421,769; and McCutcheon's Detergents and Emulsifiers, North American Edition, pages 317-324 (1986).

### Other Optional Ingredients

The present composition may optionally include one or more additional ingredients commonly used in cosmetic compositions (e.g., colorants, skin care actives, anti-inflammatory agents, sunscreen agents, emulsifiers, buffers, rheology modifiers, combinations of these and the like), provided that the additional ingredients do not undesirably alter the skin health or appearance benefits provided by the present compositions. The additional ingredients, when incorporated into the composition, should be suitable for use in contact with human skin tissue without undue toxicity, incompatibility, instability, allergic response, and the like. Some nonlimiting examples of additional actives include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

When including optional ingredients in the compositions herein, it may be desirable to select ingredients that do not form complexes or otherwise undesirably interact with other ingredients in the composition, especially pH sensitive ingredients like niacinamide, salicylates and peptides. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

### Method of Use

The present method includes identifying a target portion of skin where treatment is desired and applying a composition comprising an effective amount of bioactive materials which stimulate mitophagy and, optionally, one or more additional skin care actives to the target portion of skin. The target portion of skin may be on a facial skin surface such as the forehead, perioral, chin, periorbital, nose, and/or check) or another part of the body (e.g., hands, arms, legs, back, chest). The person or target portion of skin in need of treatment may be one that exhibits a telltale sign of aging skin (e.g., fine lines, wrinkles, hyperpigmented spots). In some instances, a target portion of skin may not exhibit a sign of skin aging, but a user may still wish to treat the portion of skin if it is one that is known to exhibit visible signs of aging (e.g., skin that is exposed to the sun). In this way, the present methods and compositions may be used prophylactically to help delay the visible signs of skin aging.

The composition may be applied to a target portion of skin and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening, and/or before bed. The treatment period herein is ideally of sufficient time for the bioactive materials to improve the appearance of the skin. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., a wrinkle or portion thereof) while minimizing delivery to skin surfaces where treatment is not desired. The composition may be applied and lightly massaged into an arca of skin. The form of the composition or the dermatologically acceptable carrier should be selected to facilitate localized application. While certain embodiments herein contemplate applying a composition locally to an area, it will be appreciated that compositions herein can be applied more generally or broadly to one or more skin surfaces. In certain embodiments, the compositions herein may be used as part of a multi-step beauty regimen, wherein the present composition may be applied before and/or after one or more other compositions.

### METHODS

### Method 1: Cell Culture

Primary human cell lines used: neonatal dermal fibroblasts (HDF434, Invitrogen, C0045C); young or aged adult, human dermal fibroblasts purchased or isolated in house from surplus human skin as previously described (Hill et al.) following informed consent.

### Method 2: Induction of Senescence

To demonstrate the ability of bioactive materials which stimulate p62-dependent mitophagy to reverse cellular aging and stress induced senescence phenotypes, human dermal fibroblasts (HDFs) were subjected to 20Gy, 10Gy, 5Gy, and 1Gy of X-ray irradiation (IR) using an X-Rad 225 irradiator (Precision X-Ray).

### Method 3: Luciferase-p62 Assay

To identify p62-dependent materials which increase mitophagy, a Luciferase-p62 Assay was established. This method provides an approach to measure the ability of a material to modulate p62-dependent mitophagy by using an inducible firefly luciferase-p62 (Fluc-p62) reporter cell system. In this assay, p62 levels are driven by doxycycline treatment. After levels increased, expression of Fluc-p62 is turned off by removal of doxycycline, materials are then added and assessed for how they affect clearance of Fluc-p62 protein, which is proportionate to cellular mitophagy rates (Kelly et al.). The negative control is doxycycline induced Luc-p62 in the absence of compound or bioactive material treatment and in the presence of vehicle (DMSO).

### Summary of Luciferase-p62 Assay

Test materials including bioactive materials were screened in mouse embryonic fibroblasts (MEFs, Harada et al.) expressing the TET-inducible p62-fluc construct as previously described (Brown et al.). The construct was generated using the doxycycline inducible pCW57.1 backbone purchased from Addgene (41393). Gibson assembly was used to insert firefly luciferase (Fluc) and p62 into the pCW57.1 backbone. Fluc, p62, and pCW57.1 were polymerase chain reaction (PCR) amplified using pfu polymerase (Life technologies) and Gibson assembly primers with 20 base pair (bp) overhangs. PCR products were separated by agarose gel electrophoreses, and fragments excised and purified before use in DNA assembly reaction with NEBuilder HiFi DNA Assembly kit (New England Biolabs). After the assembly, the reaction mix was transformed into NEB3040 Stable Competent Escherichia coli (New England Biolabs). After 24 hours growth at 31 degrees Celsius the plasmid was extracted using QIAprep Spin Miniprep kit (Qiagen). The plasmid was sequenced to confirm correct insertion of Fluc-p62. To generate MEFs stably expressing doxycycline inducible Fluc-p62, HEK293FT cells were transfected with Fluc-p62-pCW57.1 and the ViraPower Packaging Mix (Invitrogen) to produce lentivirus. MEFs were transduced with the obtained lentiviral stock following followed by selection with 1 microgram per milliliter puromycin.Fluc-p62expressing MEFs were maintained in DMEM supplemented with 10 percent fetal bovine serum (FBS), 100 units per milliliter penicillin/streptomycin, and 2 millimolar L-glutamine in a humidified atmosphere containing 5 percent carbon dioxide at 37 degrees Celsius. Cells were plated into white 96 well plates (2,000 cells per well with 100 microliters media) and allowed to settle for 24 hours. Next, the MEFs were treated with 1 microgram per milliliter doxycycline (Sigma-Aldrich 33429) for 24 hours before being rinsed 3 times with phosphate-buffered saline (PBS). Subsequently, cells were treated with test materials for 48 hours in normal growth medium and analysed using ONE-Glo™+Tox Luciferase Reporter and Cell Viability Assay (Promega) by following the manufacturer's protocol.

### Method 4: Mitochondrial Fractionation

Cells were seeded in 10 cm dishes (5 dishes per condition) and collected with ice-cold PBS by centrifugation for 5 min at 800 g at 4° C. Cells were then resuspended in 1 mL fractionation buffer (20 mM HEPES-KOH PH 7.6 (Sigma-Aldrich), 220 mM mannitol (Sigma-Aldrich), 70 mM sucrose (Sigma-Aldrich), 1 mM EDTA (Sigma-Aldrich), 2 mM DTT (Thermo Fisher Scientific) and 0.5 mM PMSF (Sigma-Aldrich)) and homogenized with 50 strokes using a dounce homogenizer (Thermo Fisher Scientific). Cell homogenates were centrifuged for 5 min at 800 g at 4° C. to pellet cellular nuclei and membrane debris. Supernatant was centrifuged again for 5 min at 800 g at 4° C. This step was repeated until no more pellet was present. Small volume (45 mL) of cleared lysate was collected as whole cell lysate, and the rest of lysate were centrifuged for 10 min at 16,100 g at 4° C. Supernatant (45 mL) was collected as cytoplasmic fractions, and mitochondria-enriched pellet was washed twice with 1 mL fractionation buffer by centrifugation for 10 min at 16,100 g at 4° C. The resulting pellet was resuspended in 450 mL fractionation buffer (10-times dilution) and subjected to immunoblot analysis.

### Method 5: Fluorescence Microscopy

Cells seeded in a 35 mm glass bottom dish (MatTek) were stained with 2.5 mM MitoSOX for 1 h and washed three times with cell culture medium. Cells were co-stained with 100 nM

Mitotracker Green or 1 mM HaloTag ligand Oregon Green. Halo-tagged p62 constructs in HeLa PentaKO cells were stained with 40 nM Janelia Fluor 646 Halo ligand for 30 min and washed three times with cell culture medium. Co-localization analysis was performed in Fiji/ImageJ with a macro to remove background followed by calculation of Manders' coefficient. To measure membrane potential, cells were stained with 16.7 nM Tetramethylrhodamine and 100 nM Mitotracker Deep Red for 30 min. CM-H2DCFDA staining was performed according to the manufacturer's instructions. Fluorescence images were obtained using an LSM700 confocal microscope (Zeiss) and analysis software (Zen 2011, Zeiss), an inverted DM5500 microscope (Leica) or an inverted DMi8 microscope (Leica) with a Plan-Apochromat 63x/1.40 oil immersion lens, equipped with an ORCA-Flash4v2.0 camera (Hamamatsu). Images were deconvolved using Huygens Essential software (version 20.10, Scientific Volume Imaging). Images were analysed in Fiji/ImageJ (version 1.48), and quantification was performed on at least 50 cells per condition. Fluorescence intensity was analyzed as outlining single cells as regions of interest and calculation of the raw integrated density value per cell. In autophagy flux assay using mRFP-GFP-LC3 reporter, the number of autophagosomes (GFP+ RFP+ puncta) and autolysosomes (GFP− RFP+ puncta) per cell were quantified by outlining single cells as regions of interest.

### Method 6: Mitophagy Assays

Mitophagy assays were performed to both demonstrate the ability of bioactive materials to restore functional mitophagy in HDFs from aged donors, and to prevent stress-associated decrease in mitophagy. Primary human dermal fibroblasts (HDFs) were cultured in DMEM supplemented with 10 percent FBS, 100 units per milliliter penicillin/streptomycin, and 2 millimolar L-glutamine in a humidified atmosphere containing 5 percent carbon dioxide at 37 degrees Celsius. Stable expression of mt-mKeima was achieved through retroviral transduction (Kelly et al.). Cells stably expressing mt-mKeima were seeded in a 35 mm glass bottom dish (MatTek). The live-cell mt-Keima signal was obtained via widefield microscopy using the DMi8 and 2 filtersets to obtain the acidic (561 nm excitation, “red”) and pH-neutral (480 nm, “green”) mKeima signals. Using FIJI/ImageJ, a macro was applied to the mt-mKeima signal to extract mitolysosomes. In detail, images were masked by applying MaxEntropy threshold to the images obtained with 561 nm excitation to remove low mt-mKeima red signal and background. Then, images were generated by subtracting the signal of green mKeima from that of red mKeima. Resulting images were binarised with the MaxEntropy threshold algorithm to extract mitolysosomes. Mitophagy events were determined as the number of puncta per cell. Cells stably expressing pSu9-Halo-GFP seeded in a 35 glass bottom dish (MatTek) were stained with 1 μM Halo TMR ligand (Promega, G8251) for 48 h. Fluorescence images were obtained using an LSM700 confocal microscope (Zeiss). The number of mitolysosomes (GFP-Halo+puncta) per cell was quantified using proprietary plugin in Fiji/ImageJ. Both mKeima and pSu9-Halo-GFP reporters directly visualize frequency of mitophagy events (e.g., mitochondria delivered by autophagolysosomes to lysosomes for degradation) (Kelly et al.).

### Method 7: Immunofluorescence

To demonstrate the ability of bioactive materials which stimulate p62-dependent mitophagy to prevent or rescue cellular aging phenotypes (e.g., increase in nuclear size, elevated p21 expression, and impaired cellular proliferation capacity), immunofluorescence microscopy was performed. Immunofluorescence analyses were performed as described previously (Carroll et al.). In brief, cells were seeded on coverslips in 24-well plates and fixed in 4% formaldehyde in PBS for 10 min at room temperature followed by permeabilization in 0.5% Triton X-100 (Sigma-Aldrich) in PBS for 4 min at room temperature (Ki67 and p21) or in methanol for 4 min at −20° C. (LC3 and p62). Cells were then blocked for 1 h in 5% normal goat serum (Sigma-Aldrich) in PBS at room temperature and incubated with primary antibodies overnight at 4° C. Cells were washed three times and incubated with the appropriate secondary antibodies for 1 h at room temperature (Thermo Fisher Scientific, A31556 and A21235, 1:1000). Cells were washed, and coverslips were mounted on slides with Prolong Gold antifade mountant with DAPI (Invitrogen). Fluorescence images were obtained as described above. The following primary antibodies were used: rabbit anti-Ki67 (Abcam, ab 15580, 1:250), rabbit anti-p21 (CST, 2947, 1:1000), mouse anti-LC3 (NanoTools, 0260-100, 1:200) and rabbit anti-p62 (MBL, PM045, 1:500).

### Method 8: SA β-Galactosidase assay

Primary human dermal fibroblasts (HDFs) were seeded on coverslips in 24-well plates. Cells were fixed with 4% formaldehyde in PBS for 10 min. Fixed cells were washed three times with PBS, before adding to each well 0.5 ml of prewarmed X-Gal staining solution (2 mM MgCl₂, 5 mM K₄Fe(CN)₆·3H₂O, 5 mM K3Fe(CN)₆, 1 mg/ml X-Gal solution ready to use (R0941, Thermo Fisher Scientific) in PBS). Plates were incubated for 24 h at 37° C., washed, mounted and imaged. SA-β-Gal activity positive and negative cells were quantified using FIJI/ImageJ. The ability of bioactive materials which increase mitophagy levels in HDFs to prevent cellular senescence was further evaluated in 6-well plates by treating with 400 micromolar hydrogen peroxide (H₂O₂) for 2 hours to introduce oxidative stress, then treated in triplicates with bioactive materials followed by incubation for 22 hours at 37 degrees Celsius, 5 percent carbon dioxide (CO₂) followed by S-β-Gal staining.

### Method 9: Cell Motility Assay

To demonstrate the ability of bioactive materials which stimulate p62-dependent mitophagy to rescue cellular aging phenotypes, improvement in cell motility was measured in primary HDFs from young and aged donors. HDFs were seeded in glass bottomed multiwell plates (Greiner, 662892), 24 h prior to imaging. Images were captured using a Zeiss CellDiscoverer 7 with a 5x/0.35NA lens with a 2× optovar using oblique mode brightfield with a Hamamatsu Fusion camera every 300 seconds for 5 h (1 ms exposure time), capturing 2 random fields per well. Cells were maintained at 37° C., 5% CO₂ throughout experiments. 6 cells were analysed per field using the Manual Tracking plugin in FIJI/ImageJ, and shown as mean movement per cell recorded.

### Method 10: Assessment of the Senescence-Associated Secretory Phenotype (SASP)

To demonstrate the ability of bioactive materials which stimulate p62-depedent mitophagy to mitigate stress-associated increases in levels of SASP factors, and to decrease levels of SASP factors associated with aging, measurement of cytokines was performed. Quantibody Human Cytokine Arrays for 20 cytokines (RayBiotech; QAH-CYT-1) were performed using conditioned media collected from primary dermal fibroblasts culture. mRNA levels of human IL-6 and IL-8 were measured by quantitative polymerase chain reaction (qPCR. Total RNA was extracted using RNeasy Mini Kit (QIAGEN, 74104). From 500 ng of total RNA, first-strand complementary DNA (cDNA) was produced using SuperScript III reverse transcriptase (Invitrogen, 18080044). Quantitative PCR was performed in a StepOnePlus Real-Time PCR system (Applied Biosystems) using Power SYBR Green PCR Master Mix (Applied Biosystems, 4367659). mRNA levels were determined with the AACt method and normalised to GAPDH levels.

### Method 11: Seahorse Analysis

To demonstrate the ability of bioactive materials which stimulate p62-dependent mitophagy to restore aging-associated decline in mitochondrial mass and function, Seahorse analysis was performed on primary HDFs from young and aged donors. The Seahorse XF Cell Mito Stress Test Kit (Agilent) was used to measure oxygen consumption rates in adult dermal fibroblasts. One day prior to the assay cells were seeded at either 4000 per well or 3000 per well. Where indicated, fibroblast plates were subjected to 20Gy IR 2 h before assay. STOCKIN (30 mM) or vehicle (DMSO) treatments were performed 7 h before Seahorse assays. HDFs were stained with 100 nM Mitotracker Green for mitochondrial density measurement. The Mito Stress Test was performed as per manufacturer's guidelines on a Seahorse XF96 Extracellular Flux analyser using previously optimized conditions. Following assay completion, cells were fixed in 4% PFA for 20 minutes. PFA was then removed, and cells were washed twice with PBS. Following fixation, cells were stained with Hoescht nucleic acid stain (Invitrogen) for 10 min before being washed twice in PBS. Fluorescence was subsequently measured using the PHERAstar FSX plate reader (BMG LABTECH) at 350/460 nm for normalization purposes. Following normalization, OCR measurements were interpreted from data generated from the Agilent Seahorse Wave software version 2.6.3.5. Basal respiration data in unstressed conditions is presented. OCR measurements were further normalised to mitochondrial density to obtain ATP production (pmol/min/mitochondria).

### Method 12: Statistical Analyses

Graphical data denote the mean±s.e.m. (of n=3 or more biological replicates) and are depicted by column graph scatter dot plot, or displayed as cell popular violin plots using Prism 8.4.3 software (GraphPad). P values were determined by Student's t test (two-tailed, unpaired) between two groups, one-way or two-way ANOVA, followed by Dunnett's or Sidak's post-hoc analysis, or multiple t-test with false discovery rate approach using two-stage linear step-up procedure of Benjamini, Krieger, and using Prism 8.4.3 software (GraphPad), unless otherwise stated. A P value<0.05 was considered significant. *, P<0.05; **, P<0.01; ***, P<0.001; ****, P<0.0001; ns (non-significant).

### Method 13: Docking Experiments of Steranes

For in silico docking simulations molecules were docked to the binding pocket of p62 ZZ domain (PDB ID: 6MJ7, resolution 1.448 A) (Zhang et al.) which was obtained from the Protein Data Bank (PDB). Maestro (Schrodinger 2023) was used to prepare the crystal structure for docking and the co-crystalized ligand was removed. The grid of the binding pocket was generated to include the ligand binding residues (i.e. N125, 1127, D129, N132, R139 and D149).

### EXAMPLES

### Example 1: Formulations

Table 1 below provides examples of the present skin care compositions, with formulations I, II, III, IV, V, VI, VII, VIII, and IX representing comparative formulations, and formulations X, XI, XII, XIII, XIV, XV, XVI, XVII, XVIII, and XIX representing inventive formulations. The exemplary compositions are made by blending the A phase components with a suitable mixer (e.g., Tekmar RW20DZM or equivalent) and heating to a temperature of 70-80° C. and maintaining the temperature while stirring. Separately, the B phase components are blended with a suitable mixer and heated to 70-75° C., while maintaining temperature during mixing. Phase B is added to Phase A while mixing well to form an oil-in-water (O/W) emulsion. The emulsion is then milled using a suitable mill (e.g., Tekmar T-25 or equivalent) for 5 minutes. When the emulsion is at 60° C., phase C is added while continuing to mix. At 40° C., the ingredients of phase D and E are added to the emulsion. The emulsion is then milled for 5 minutes to provide a uniform composition.

TABLE 1

| Component | I | II | III | IV | V | VI | VII | VIII | IX |
|---|---|---|---|---|---|---|---|---|---|
| Phase A | | | | | | | | | |
| Water | qs | qs | qs | qs | qs | qs | qs | qs | qs |
| Glycerol | 5.00 | 7.00 | 3.00 | 15.0 | 7.00 | 5.00 | 5.00 | 3.00 | 5.00 |
| Disodium EDTA | 0.10 | 0.05 | 0.10 | 0.10 | 0.05 | 0.05 | 0.05 | 0.05 | 0.10 |
| Phase B | | | | | | | | | |
| Dimethicone 5 cSt | — | — | — | — | — | — | — | 10.0 | 15.0 |
| Dimethicone and | — | — | — | — | — | — | — | 13.0 | 15.0 |
| Dimethicone | | | | | | | | | |
| Crosspolymer | | | | | | | | | |
| Laureth-4 | — | — | — | — | — | — | — | 0.25 | 0.35 |
| Polysorbate 20 | — | — | — | — | — | — | — | 0.15 | 0.25 |
| Tapioca Starch and | — | — | — | — | — | — | — | 2.50 | 3.50 |
| Polymethylsilsesquioxane | | | | | | | | | |
| Avobenzone | — | — | — | 3.00 | — | 3.00 | — | — | — |
| Homosalate | — | — | — | 15.0 | — | 10.0 | — | — | — |
| Octisalate | — | — | — | 5.00 | — | 5.00 | — | — | — |
| Octocrylene | — | — | — | 2.60 | — | 9.00 | — | — | — |
| Isopropyl Isostearate | 5.00 | 2.50 | 1.00 | — | — | — | — | — | — |
| Isohexadecane | 1.00 | 1.50 | 3.00 | — | — | — | — | — | — |
| Cetyl Alcohol | 0.25 | 0.50 | 0.32 | 0.40 | 0.40 | 0.30 | 0.50 | — | — |
| Tocopherol Acetate | | 0.50 | 0.25 | 1.00 | 0.25 | 0.25 | 0.25 | — | — |
| PEG-100 Stearate | 0.20 | 0.10 | 0.10 | 0.30 | 0.10 | 0.20 | 0.10 | — | — |
| Stearyl Alcohol | 0.50 | 1.50 | 0.40 | 0.60 | 0.50 | 0.40 | 0.60 | — | — |
| Behenyl Alcohol | 0.40 | 1.00 | 0.50 | 0.50 | 0.40 | 0.35 | 0.50 | — | — |
| Ethyl Paraben | 0.20 | 0.15 | 0.20 | 0.25 | — | — | — | — | — |
| Propyl Paraben | 0.10 | 0.15 | 0.10 | 0.15 | — | — | — | — | — |
| Polymethylsilsesquioxane | 1.25 | 2.50 | 1.00 | — | — | — | — | — | — |
| Phase C | | | | | | | | | |
| Titanium Dioxide | — | 0.50 | — | 0.25 | — | — | — | — | — |
| Tapioca Starch and | — | — | — | — | — | 12.0 | | — | — |
| Polymethylsilsesquioxane | | | | | | | | | |
| Vinyl | 1.50 | | 1.50 | 3.50 | 5.00 | — | 7.50 | — | — |
| Dimethicone/Methicone | | | | | | | | | |
| Silsesquioxane | | | | | | | | | |
| Crosspolymer | | | | | | | | | |
| Sodium Polyacrylate | — | — | — | — | 1.50 | 1.00 | 1.50 | — | — |
| Starch | | | | | | | | | |
| Hydroxyethyl | 2.00 | 1.50 | 2.50 | 2.00 | — | — | — | 1.25 | 2.00 |
| acrylate/sodium | | | | | | | | | |
| acryloyldimethyltaurate | | | | | | | | | |
| copolymer | | | | | | | | | |
| Phase D | | | | | | | | | |
| Water | 5 | 10 | 10 | 5 | 10 | 10 | 10 | 5 | 10 |
| Pal-KT | 0.00005 | 1 | 0.5 | 0.1 | 0.05 | 0.025 | 0.01 | 0.005 | 0.0005 |
| Ac-PPYL | 0.00005 | 1 | 5 | 1 | 0.1 | 0.005 | 0.05 | 0.0005 | 0.005 |
| Niacinamide | 3.5 | — | 3.5 | — | 4 | 5 | — | — | 2 |
| Dexpanthenol | 0.5 | 0.5 | 0.5 | 1 | 1 | 1.5 | 0.25 | 1 | 0.5 |
| Phase E | | | | | | | | | |
| Benzyl alcohol | 0.25 | 0.40 | 0.25 | 0.50 | — | — | — | — | — |
| Hexanediol and Caprylyl | — | — | — | — | 0.70 | 0.80 | 0.70 | 0.70 | 1.00 |
| Glycol | | | | | | | | | |
| Phenoxyethanol | — | — | — | — | 0.3 | 0.4 | 0.5 | 0.20 | 0.25 |
| Dimethicone/dimethiconol | 0.5 | 1.00 | 2.00 | 1.00 | 2.00 | 2.00 | 1.00 | 1.75 | 1.00 |

| Component | X | XI | XII | XIII | XIV | XV | XVI | XVII | XVIII | XIX |
|---|---|---|---|---|---|---|---|---|---|---|
| Phase A | | | | | | | | | | |
| Water | qs | qs | qs | qs | qs | qs | qs | qs | qs | qs |
| Glycerol | 5.00 | 7.00 | 3.00 | 15.0 | 7.00 | 5.00 | 5.00 | 3.00 | 5.00 | 7.00 |
| Disodium EDTA | 0.10 | 0.05 | 0.10 | 0.10 | 0.05 | 0.05 | 0.05 | 0.05 | 0.10 | 0.05 |
| Phase B | | | | | | | | | | |
| Dimethicone 5 cSt | — | — | — | — | — | — | — | 10.0 | 15.0 | — |
| Dimethicone and | — | — | — | — | — | — | — | 13.0 | 15.0 | — |
| Dimethicone Crosspolymer | | | | | | | | | | |
| Laureth-4 | — | — | — | — | — | — | — | 0.25 | 0.35 | — |
| Polysorbate 20 | — | — | — | — | — | — | — | 0.15 | 0.25 | — |
| Tapioca Starch and | — | — | — | — | — | — | — | 2.50 | 3.50 | — |
| Polymethylsilsesquioxane | | | | | | | | | | |
| Avobenzone | — | — | — | 3.00 | — | 3.00 | — | — | — | — |
| Homosalate | — | — | — | 15.0 | — | 10.0 | — | — | — | — |
| Octisalate | — | — | — | 5.00 | — | 5.00 | — | — | — | — |
| Octocrylene | — | — | — | 2.60 | — | 9.00 | — | — | — | — |
| Isopropyl Isostearate | 5.00 | 2.50 | 1.00 | — | — | — | — | — | — | 5.00 |
| Isohexadecane | 1.00 | 1.50 | 3.00 | — | — | — | — | — | — | 1.00 |
| Cetyl Alcohol | 0.25 | 0.50 | 0.32 | 0.40 | 0.40 | 0.30 | 0.50 | — | — | 0.25 |
| Tocopherol Acetate | — | 0.50 | 0.25 | 1.00 | 0.25 | 0.25 | 0.25 | — | — | |
| PEG-100 Stearate | 0.20 | 0.10 | 0.10 | 0.30 | 0.10 | 0.20 | 0.10 | — | — | 0.20 |
| Stearyl Alcohol | 0.50 | 1.50 | 0.40 | 0.60 | 0.50 | 0.40 | 0.60 | — | — | 0.50 |
| Behenyl Alcohol | 0.40 | 1.00 | 0.50 | 0.50 | 0.40 | 0.35 | 0.50 | — | — | 0.40 |
| Polymethylsilsesquioxane | 1.25 | 2.50 | 1.00 | — | — | — | — | — | — | 1.25 |
| Isopropyl Lauroyl | 3.0 | 5.0 | 1.0 | — | — | — | — | — | 5.0 | — |
| Sarcosinate | | | | | | | | | | |
| Pentylene Glycol | 3.0 | 3.0 | 1.0 | — | — | — | — | — | 3.0 | — |
| Magnolol | 0.10 | — | — | — | — | — | — | — | — | — |
| Carnosic Acid | — | 0.3 | — | — | — | — | — | — | — | — |
| Ursolic acid | — | — | 0.3 | — | — | — | — | — | — | — |
| Xanthohumol | — | — | — | 0.5 | — | — | — | — | — | — |
| Oxyresveratrol | — | — | — | — | 0.25 | — | — | — | — | — |
| Hydroxytyrosol | — | — | — | — | — | 0.25 | — | — | — | — |
| Gallic acid | — | — | — | — | — | — | 0.25 | — | — | — |
| Phloretin | — | — | — | — | — | — | — | 0.5 | — | — |
| p-Coumaric Acid | — | — | — | — | — | — | — | — | 1.0 | — |
| Phase C | — | — | — | — | — | — | — | — | — | — |
| Titanium Dioxide | — | 0.50 | — | 0.25 | — | — | — | — | — | — |
| Tapioca Starch and | — | — | — | — | — | 12.0 | | — | — | — |
| Polymethylsilsesquioxane | | | | | | | | | | |
| Vinyl | 1.50 | | 1.50 | 3.50 | 5.00 | — | 7.50 | — | 3.5 | — |
| Dimethicone/Methicone | | | | | | | | | | |
| Silsesquioxane Crosspolymer | | | | | | | | | | |
| Sodium Polyacrylate Starch | — | — | — | — | 1.50 | 1.00 | 1.50 | — | — | — |
| Hydroxyethyl | 2.00 | 1.50 | 2.50 | 2.00 | — | — | — | 1.25 | 2.00 | — |
| acrylate/sodium | | | | | | | | | | |
| acryloyldimethyltaurate | | | | | | | | | | |
| copolymer | | | | | | | | | | |
| Phase D | | | | | | | | | | — |
| Water | 5 | 10 | 10 | 5 | 10 | 10 | 10 | 5 | 10 | 10 |
| Niacinamide | 3.5 | — | 3.5 | — | 4 | 5 | — | — | 2 | 2 |
| Dexpanthenol | 0.5 | 0.5 | 0.5 | 1 | 1 | 1.5 | 0.25 | 1 | 0.5 | 0.5 |
| Punicalagin | — | — | — | — | — | — | — | — | — | 2.0 |
| Phase E | | | | | | | | | | |
| Benzyl alcohol | 0.25 | 0.40 | 0.25 | 0.50 | — | — | — | — | — | 0.50 |
| Sodium Benzoate | — | — | — | — | — | — | — | — | — | 0.10 |
| Hexanediol and Caprylyl | — | — | — | — | 0.70 | 0.80 | 0.70 | 0.70 | 1.00 | — |
| Glycol | | | | | | | | | | |
| Phenoxyethanol | — | — | — | — | 0.3 | 0.4 | 0.5 | 0.20 | 0.25 | 0.5 |
| Dimethicone/dimethiconol | 0.5 | 1.00 | 2.00 | 1.00 | 2.00 | 2.00 | 1.00 | 1.75 | 1.00 | 0.5 |

In one embodiment the skin care compositions are those described in the formulations shown in X, XI, XII, XIII, XIV, XV, XVI, XVII, XVIII, or XIX, as shown in Table 1.

### Example 2. Bioactive Materials can Increase p62-Dependent Mitophagy Levels and Reverse Cellular Aging Phenotypes in Skin Cells from Aged Donors, Which Exhibit an Impairment in Functional Mitophagy.

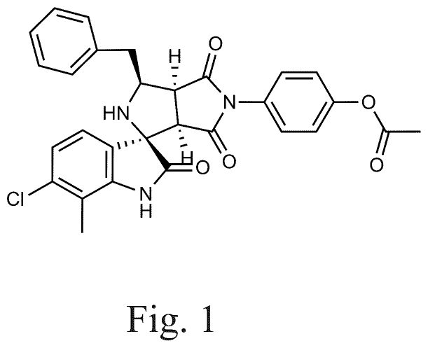

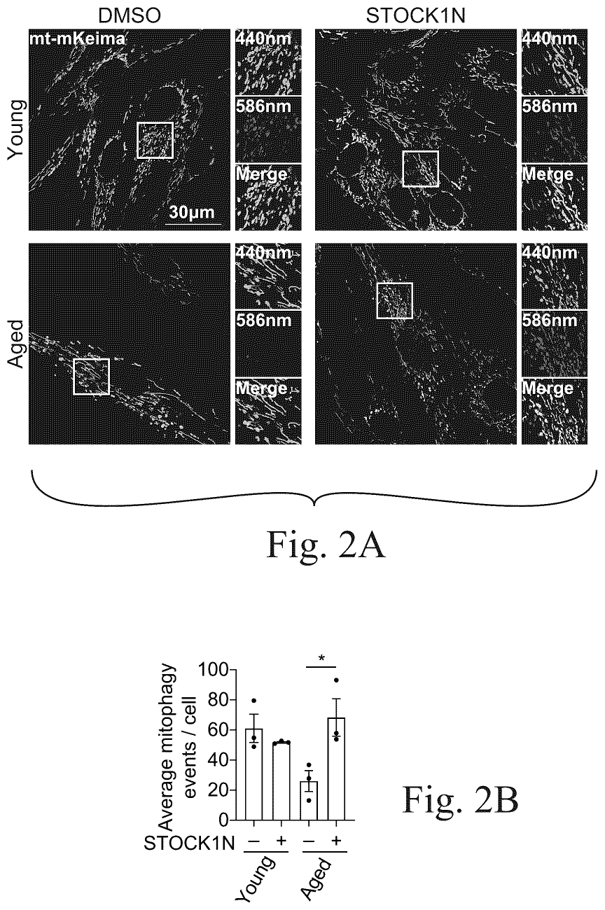

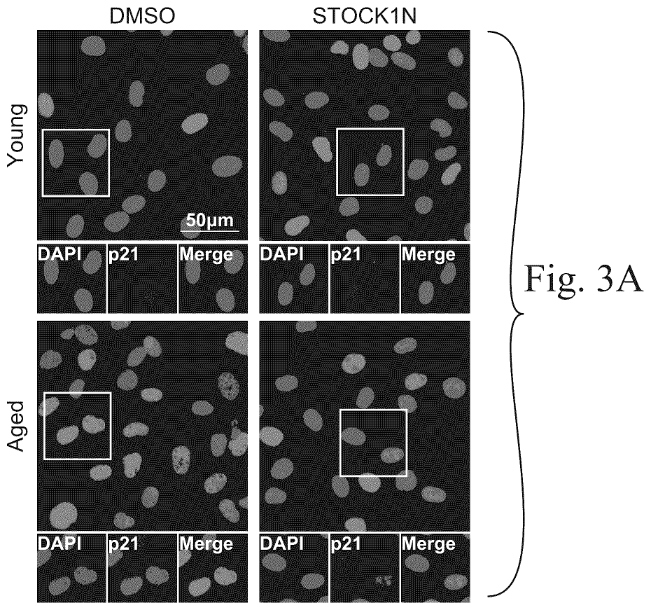

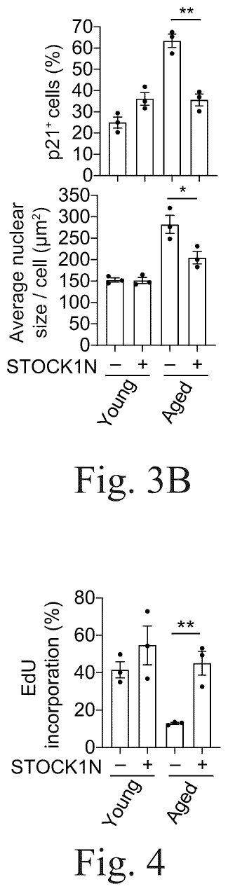

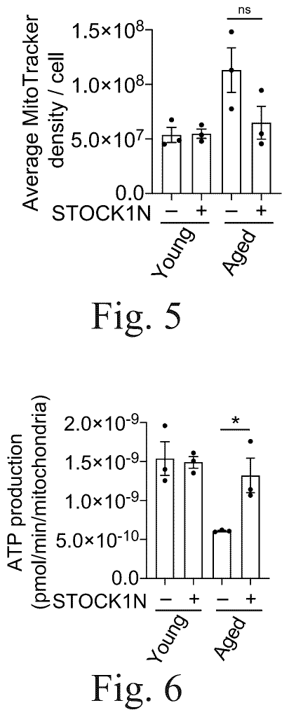

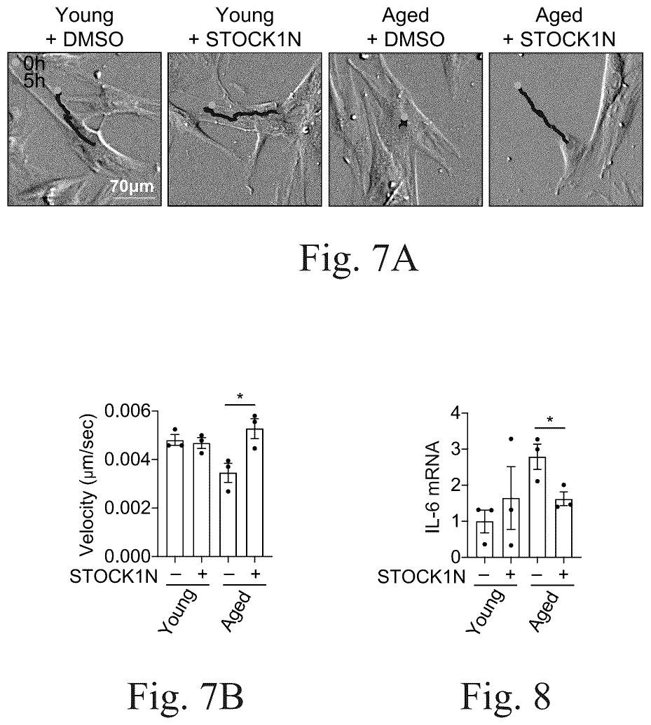

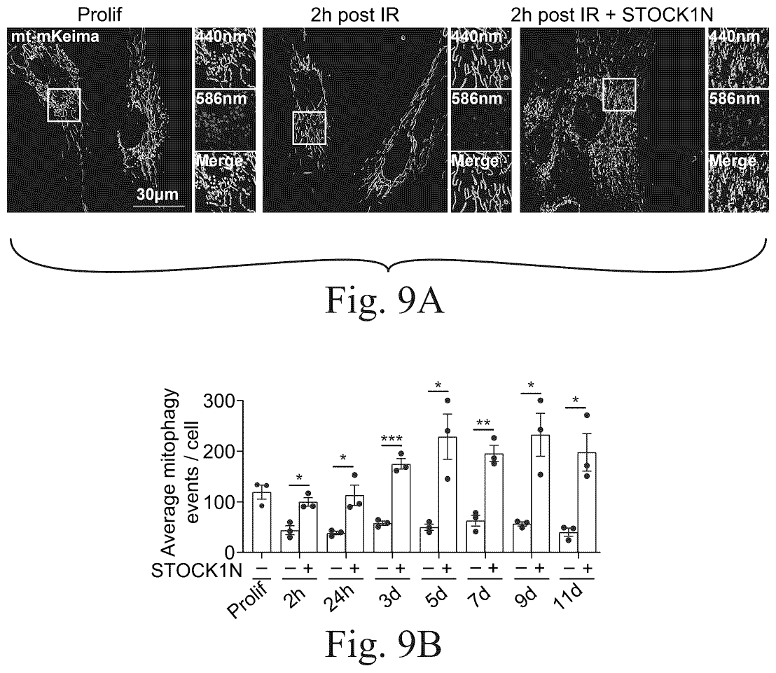

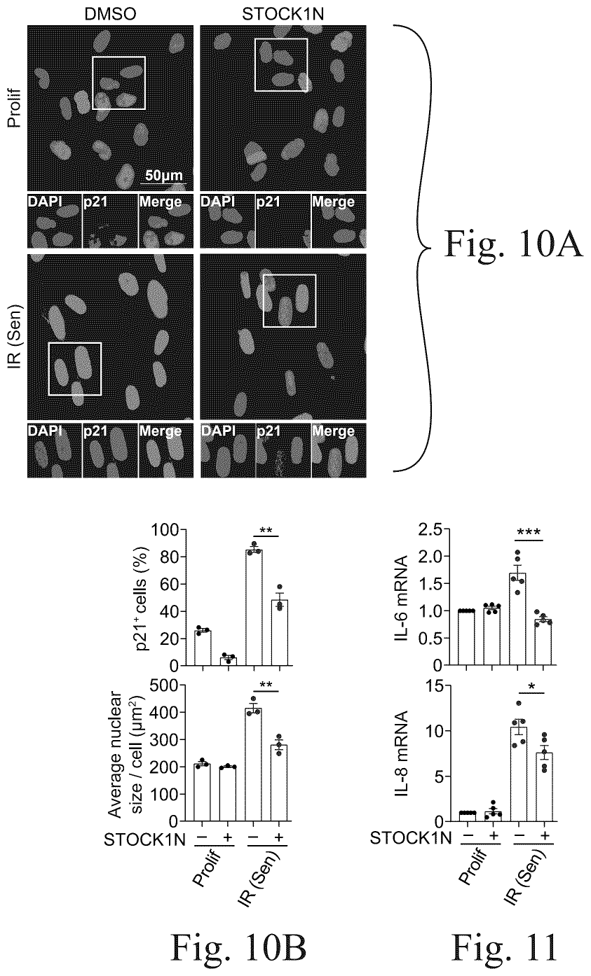

This example demonstrates the ability of a reference compound (STOCKIN-57543) (FIG. 1) to restore p62-dependent mitophagy levels in skin cells from aged donors and rescue cellular senescence and aging phenotypes. Validating the desired result with the reference compound STOCKIN-57543 demonstrates a “threshold” of p62 activity required to prevent/reverse aging, thus leading to the rationale that other compounds that perform as well or better than STOCKIN would be expected to have the same effect. With informed consent, human dermal fibroblasts (HDFs) were isolated from young (≤29 years old) and aged (≥59 years old) donors, with 3 from each age group, Kelly et al. STOCKIN-57534 treatment of HDFs at 10 μM resulted in a 33% increase p62-dependent mitophagy levels over baseline in the Luciferase-p62 assay. Furthermore, STOCKIN-57534 was shown to restore functional mitophagy via a p62-dependent mechanism as it was unable to rescue the suppression of basal mitophagy in the absence of p62 (resulting from p62 knockdown) in HDFs (Kelly et al.). STOCKIN-57534 was additionally demonstrated to reverse cellular aging and senescence phenotypes in HDFs, including: restoring functional mitophagy (FIGS. 2A and 2B) (Method 6), decreasing elevated p21 expression (FIG. 3 (FIGS. 3A and 3B)) (Method 7), decreasing a senescence-associated increase in nuclear size (FIG. 3) (Method 7), restoring cellular proliferation capacity (FIG. 4), restoring mitochondrial mass (FIG. 5) (Method 11) and function (FIG. 6) (Method 11), improving cell motility (FIGS. 7A and 7B) (Method 9), and decreasing levels of the SASP factor IL-6 (FIG. 8) (Method 10).

By validating STOCKIN-57534 as a p62-dependent mitophagy activator in skin cells and demonstrating its ability to rescue cellular senescence and aging phenotypes in cells from aged donors in which basal mitophagy is suppressed, we have thereby established that a 33% increase in p62-dependent mitophagy levels by a compound, for example, a bioactive material, tested at 10 μM in the Luciferase-p62 assay correlates with the ability of the compound to reverse cellular senescence and aging phenotypes. Thus, it is logical to theorize that other compounds or bioactive materials tested at the same concentration which deliver a 33% or greater response in the Luciferase-p62 assay would also deliver meaningful cellular anti-aging benefits in skin cells from aged donors. We therefore employed the Luciferase-p62 screening assay to identify additional novel mitophagy activators which can reverse cellular aging and senescence phenotypes.

### Example 3. Bioactive Materials can Increase p62-dependent Mitophagy Levels in Skin Cells Under Stress Conditions in Which Mitophagy is Suppressed and Prevent Cellular Senescence and Aging Phenotypes

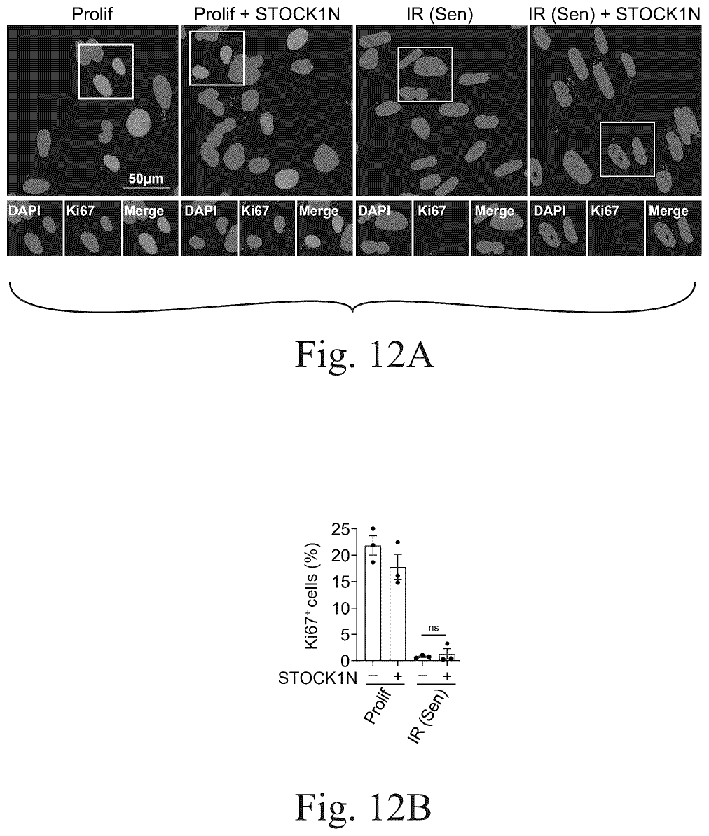

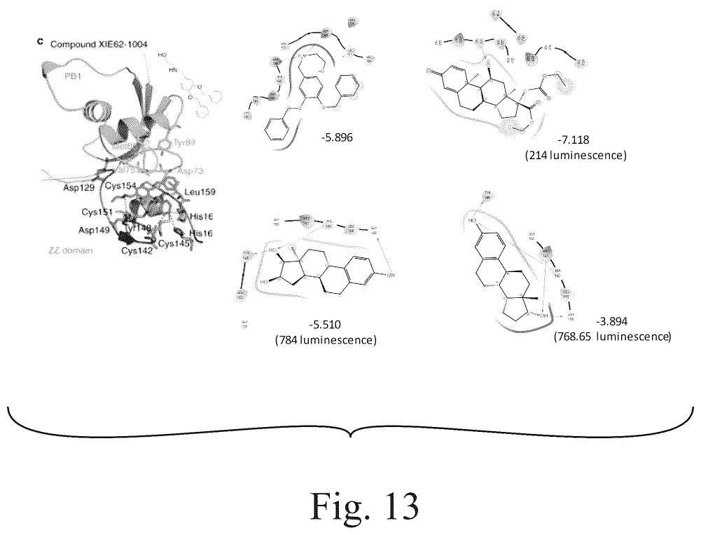

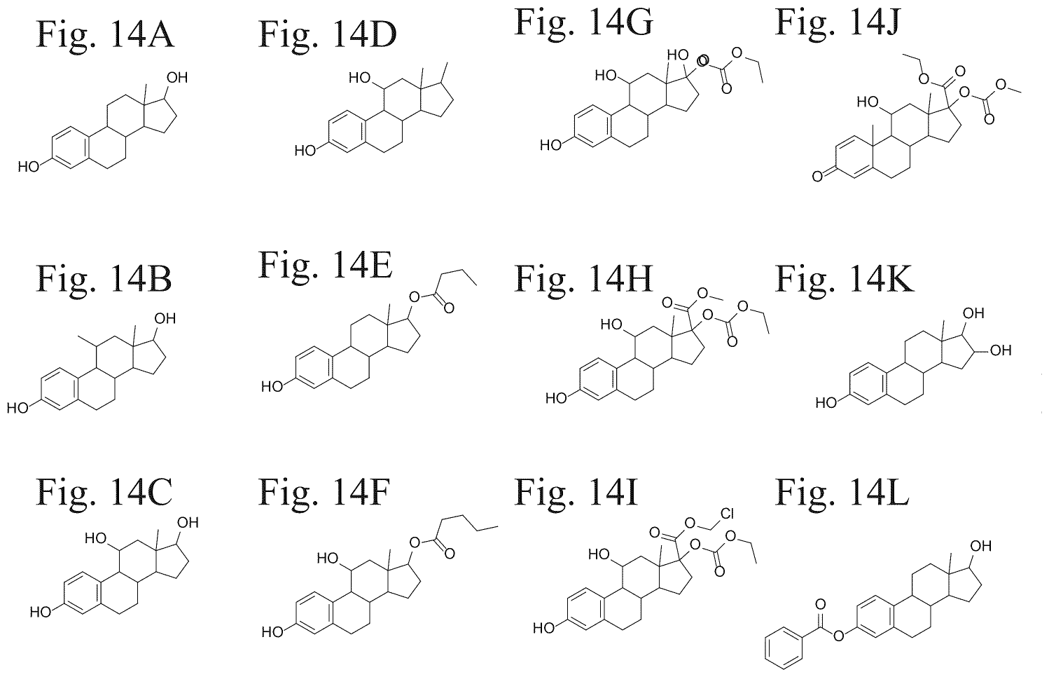

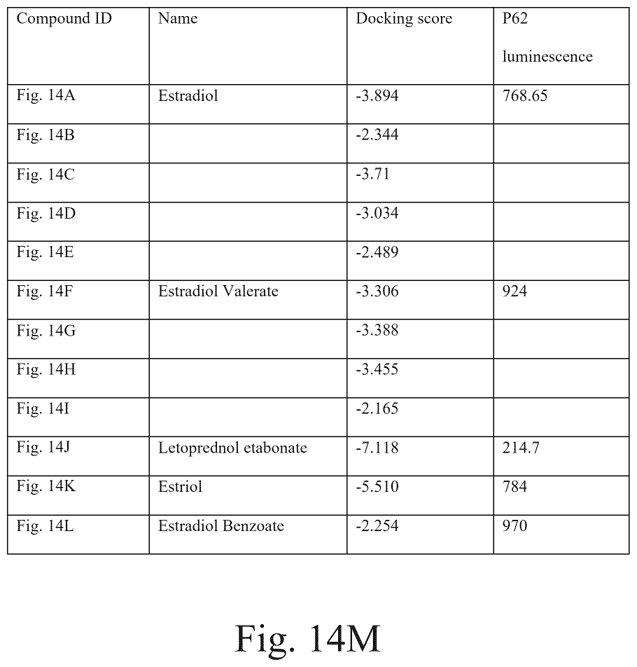

This example demonstrates the ability of a reference compound (STOCKIN-57543) to increase p62-dependent mitophagy in skin cells subjected to irradiation (IR), thereby preventing cellular senescence phenotypes induced by a failure in functional mitophagy under these stress conditions. Specifically, treatment of skin cells with STOCKIN-57543 5h prior to IR exposure was demonstrated to prevent cellular aging and senescence phenotypes induced by acute cellular stress, including: preventing IR-associated decrease in mitophagy (FIG. 9) (Method 6), mitigating IR-associated increase in nuclear size (FIG. 10) (Method 7), mitigating IR-associated increased p21 expression (FIG. 10) (Method 7), mitigating IR-associated increases in levels of the SASP factors IL-6 and IL-8 (FIG. 11) (Method 10), and mitigating IR-associated decrease in cellular proliferation capacity (FIGS. 12A and 12B) (Method 7).

By demonstrating the ability of the p62-dependent mitophagy activator STOCKIN-57534 to prevent cellular senescence and aging phenotypes under IR stress conditions in which basal mitophagy is suppressed, we have thereby established that a 33% increase in p62-dependent mitophagy levels for a compound, for example, a bioactive material, tested at 10 μM in the Luciferase-p62 assay is correlated with the ability of the compound to prevent IR-induced cellular senescence and aging phenotypes. Thus, it is logical to theorize that other compounds or bioactive materials tested at the same tested concentration which deliver a 33% or greater response in the Luciferase-p62 assay would also effectively prevent cellular aging and senescence phenotypes in skin cells exposed to IR in which basal mitophagy is suppressed. We therefore employed this screening assay to identify additional novel activators of mitophagy which can prevent IR-associated cellular aging and senescence phenotypes.

### Example 4: Identification of p62-dependent Bioactive Materials Which Increase Mitophagy

This example demonstrates the abilities of 209 bioactive materials to stimulate p62-dependent mitophagy in mouse embryonic fibroblasts (MEFs). Test compositions and control compositions were prepared as described above in the Luciferase-p62 assay (Method 3) and tested accordingly. The compounds used in this example, as examples of bioactive materials, were from the APExBIO DiscoveryProbe™ Natural Product Library Plus (Houston, TX) and evaluated at equivalent concentrations (10 μM). The results of the test are summarized below (Table 2). Data are calculated as percentage of Luciferase-p62 clearance relative to baseline (mean luminescence in the absence of doxycycline induction) with a cut-off of 33% activity which was sufficient to identify bioactive materials which prevent or reverse cellular senescence and aging phenotypes under conditions of mitophagy suppression (Examples 2 and 3). As evidenced in Table 2, a total of 209 materials tested yielded the desired effect. The materials listed in Table 2 are examples of bioactive materials.

Data are additionally calculated as percentage of activity relative to rapamycin, a benchmark control compound tested in the same experiment. Rapamycin is a pharmacological inhibitor of mTOR with established senotherapeutic activity (Mannick et al.), which has been previously demonstrated to provide anti-aging benefits in human skin when delivered via a topical formulation (Chung et al.). 52 materials (listed in Table 2) were found to stimulate p62-dependent mitophagy above the response measured for rapamycin at the same tested concentration.

TABLE 2

| | | | | | % Activity |
|---|---|---|---|---|---|
| | | CAS | Mean | % Activity | relative to |
| # | Compound | Number | Luminescence | over baseline | Rapamycin |
| 1 | Loteprednol etabonate | 82034-46-6 | 214.82 | 86.72 | 156.26 |
| 2 | Tubeimoside I | 102040-03-9 | 257.38 | 84.09 | 151.52 |
| 3 | Fidaxomicin | 873857-62-6 | 262.87 | 83.75 | 150.90 |
| 4 | Chloroxine | 773-76-2 | 272.45 | 83.16 | 149.84 |
| 5 | Miconazole | 22916-47-8 | 297.40 | 81.62 | 147.06 |
| 6 | 17-AAG (KOS953) | 75747-14-7 | 299.07 | 81.52 | 146.87 |
| 7 | Cyclosporin A | 59865-13-3 | 345.35 | 78.66 | 141.72 |
| 8 | Bisdemethoxycurcumin | 24939-16-0 | 355.33 | 78.04 | 140.61 |
| 9 | Brefeldin A | 20350-15-6 | 356.17 | 77.99 | 140.52 |
| 10 | Econazole nitrate | 24169-02-6 | 359.76 | 77.77 | 140.12 |
| 11 | Oxyresveratrol | 29700-22-9 | 403.74 | 75.05 | 135.22 |
| 12 | Dasatinib Monohydrate | 863127-77-9 | 410.49 | 74.63 | 134.47 |
| 13 | 1,4-Benzoquinone | 106-51-4 | 422.02 | 73.92 | 133.18 |
| 14 | Itraconazole | 84625-61-6 | 450.47 | 72.16 | 130.02 |
| 15 | Cyclopamine | 4449-51-8 | 471.92 | 70.83 | 127.63 |
| 16 | Moxidectin | 113507-06-5 | 476.19 | 70.57 | 127.15 |
| 17 | 4-Aminophenol | 123-30-8 | 482.45 | 70.18 | 126.45 |
| 18 | Xanthohumol | 6754-58-1 | 485.82 | 69.98 | 126.08 |
| 19 | Vitamin D3 | 67-97-0 | 488.16 | 69.83 | 125.82 |
| 20 | Cyclosporine | 79217-60-0 | 497.26 | 69.27 | 124.81 |
| 21 | Tacrolimus (FK506) | 104987-11-3 | 512.17 | 68.35 | 123.15 |
| 22 | 10-Gingerol | 23513-15-7 | 520.57 | 67.83 | 122.21 |
| 23 | Clindamycin Phosphate | 24729-96-2 | 526.49 | 67.46 | 121.55 |
| 24 | Tioconazole | 65899-73-2 | 537.96 | 66.75 | 120.27 |
| 25 | Dronedarone | 141626-36-0 | 550.43 | 65.98 | 118.89 |
| 26 | Saikosaponin A | 20736-09-8 | 561.50 | 65.30 | 117.65 |
| 27 | Lapatinib Ditosylate | 388082-77-7 | 578.14 | 64.27 | 115.80 |
| 28 | L-Acetylcarnitine | 5080-50-2 | 581.20 | 64.08 | 115.46 |
| | (hydrochloride) | | | | |
| 29 | Butenafine HCl | 101827-46-7 | 582.45 | 64.00 | 115.32 |
| 30 | Deoxycholic acid | 302-95-4 | 591.12 | 63.47 | 114.35 |
| | sodium salt | | | | |
| 31 | Mycophenolate Mofetil | 128794-94-5 | 594.21 | 63.28 | 114.01 |
| 32 | Artemether (SM-224) | 71963-77-4 | 596.56 | 63.13 | 113.75 |
| 33 | Bleomycin Sulfate | 9041-93-4 | 615.38 | 61.97 | 111.65 |
| 34 | Clotrimazole | 23593-75-1 | 623.22 | 61.48 | 110.78 |
| 35 | Ascomycin(FK 520) | 104987-12-4 | 640.39 | 60.42 | 108.87 |
| 36 | Itaconic acid | 97-65-4 | 640.97 | 60.39 | 108.80 |
| 37 | Deoxyarbutin | 53936-56-4 | 653.14 | 59.64 | 107.45 |
| 38 | Carnosic acid | 3650-09-7 | 661.96 | 59.09 | 106.47 |
| 39 | ASC-J9 | 52328-98-0 | 664.12 | 58.96 | 106.23 |
| 40 | N-Ethylmaleimide | 128-53-0 | 668.28 | 58.70 | 105.76 |
| | (NEM) | | | | |
| 41 | myo-Inositol | 87-89-8 | 670.16 | 58.58 | 105.55 |
| 42 | Estrone | 53-16-7 | 670.65 | 58.55 | 105.50 |
| 43 | Dexamethasone acetate | 1177-87-3 | 681.22 | 57.90 | 104.32 |
| 44 | Bromocriptine mesylate | 22260-51-1 | 685.00 | 57.67 | 103.90 |
| 45 | Erythritol | 149-32-6 | 696.09 | 56.98 | 102.67 |
| 46 | Raspberry ketone | 5471-51-2 | 696.39 | 56.96 | 102.63 |
| 47 | Miconazole Nitrate | 22832-87-7 | 704.35 | 56.47 | 101.75 |
| 48 | 2′,5′- | 490-78-8 | 709.63 | 56.14 | 101.16 |
| | Dihydroxyacetophenone | | | | |
| 49 | Mollugin | 55481-88-4 | 709.65 | 56.14 | 101.16 |
| 50 | Protodioscin | 55056-80-9 | 713.58 | 55.90 | 100.72 |
| 51 | Melibiose | 585-99-9 | 715.78 | 55.76 | 100.47 |
| 52 | Vincamine | 1617-90-9 | 716.24 | 55.74 | 100.42 |
| 53 | Spermine | 71-44-3 | 720.40 | 55.48 | 99.96 |
| 54 | Cephalothin | 153-61-7 | 727.65 | 55.03 | 99.15 |
| 55 | m-Hydroxybenzoic acid | 99-06-9 | 745.89 | 53.90 | 97.12 |
| 56 | Limonin | 1180-71-8 | 753.03 | 53.46 | 96.33 |
| 57 | Fosaprepitant | 265121-04-8 | 756.54 | 53.24 | 95.93 |
| | dimeglumine salt | | | | |
| 58 | Sofalcone | 64506-49-6 | 760.95 | 52.97 | 95.44 |
| 59 | 2,3-Butanedione-2- | 57-71-6 | 762.32 | 52.89 | 95.29 |
| | monoxime | | | | |
| 60 | Folinic acid | 58-05-9 | 762.92 | 52.85 | 95.22 |
| 61 | D-Phenylalanine | 673-06-3 | 763.78 | 52.80 | 95.13 |
| 62 | Nicergoline | 27848-84-6 | 764.49 | 52.75 | 95.05 |
| 63 | Oleandrin | 465-16-7 | 766.18 | 52.65 | 94.86 |
| 64 | Berberrubine | 15401-69-1 | 766.18 | 52.65 | 94.86 |
| 65 | Estradiol | 50-28-2 | 768.64 | 52.50 | 94.59 |
| 66 | Levulinic acid | 123-76-2 | 771.58 | 52.32 | 94.26 |
| 67 | Tretinoin (Aberela) | 302-79-4 | 773.39 | 52.20 | 94.06 |
| 68 | Isovaleroylglycine | 16284-60-9 | 776.88 | 51.99 | 93.67 |
| 69 | Besifloxacin HCl | 405165-61-9 | 777.82 | 51.93 | 93.56 |
| 70 | Ceftiofur HCl | 103980-44-5 | 781.02 | 51.73 | 93.21 |
| 71 | α-Naphthoflavone | 604-59-1 | 781.48 | 51.70 | 93.16 |
| 72 | Punicalagin | 65995-63-3 | 782.52 | 51.64 | 93.04 |
| 73 | Estriol | 50-27-1 | 784.74 | 51.50 | 92.80 |
| 74 | Dimethyl Fumarate | 624-49-7 | 786.79 | 51.38 | 92.57 |
| 75 | Ouabain | 630-60-4 | 788.34 | 51.28 | 92.39 |
| 76 | Salinomycin | 53003-10-4 | 792.70 | 51.01 | 91.91 |
| 77 | Robenidine | 25875-50-7 | 794.31 | 50.91 | 91.73 |
| | hydrochloride | | | | |
| 78 | Mecarbinate | 15574-49-9 | 799.96 | 50.56 | 91.10 |
| 79 | Medroxyprogesterone | 71-58-9 | 800.04 | 50.56 | 91.09 |
| | acetate | | | | |
| 80 | Hydrocortisone | 50-23-7 | 801.04 | 50.50 | 90.98 |
| 81 | (S)-(+)-Carvone | 2244-16-8 | 802.50 | 50.40 | 90.82 |
| 82 | Bedaquiline | 843663-66-1 | 802.99 | 50.37 | 90.76 |
| 83 | Clindamycin HCl | 21462-39-5 | 803.11 | 50.37 | 90.75 |
| 84 | Reserpine | 16994-56-2 | 810.13 | 49.93 | 89.97 |
| | hydrochloride | | | | |
| 85 | 3-Methylglutaric acid | 626-51-7 | 818.33 | 49.43 | 89.05 |
| 86 | Andrographolide | 5508-58-7 | 818.97 | 49.39 | 88.98 |
| 87 | Mycophenolic acid | 24280-93-1 | 821.72 | 49.22 | 88.68 |
| 88 | Sulfadimethoxine | 122-11-2 | 821.82 | 49.21 | 88.67 |
| 89 | Capsaicin | 404-86-4 | 832.02 | 48.58 | 87.53 |
| 90 | Hydroxytyrosol | 10597-60-1 | 839.13 | 48.14 | 86.74 |
| 91 | Cytidine | 65-46-3 | 841.40 | 48.00 | 86.49 |
| 92 | 2,5-Dimethyl pyrazine | 123-32-0 | 842.32 | 47.94 | 86.38 |
| 93 | Oleuropein | 32619-42-4 | 843.32 | 47.88 | 86.27 |
| 94 | Pioglitazone | 111025-46-8 | 847.16 | 47.65 | 85.84 |
| 95 | Fluorouracil (Adrucil) | 51-21-8 | 848.60 | 47.56 | 85.68 |
| 96 | Cedrol | 77-53-2 | 850.22 | 47.46 | 85.50 |
| 97 | Eriodictyol | 552-58-9 | 852.50 | 47.31 | 85.25 |
| 98 | Carbenoxolone | 7421-40-1 | 856.44 | 47.07 | 84.81 |
| | disodium | | | | |
| 99 | Bacitracin | 1405-87-4 | 860.05 | 46.85 | 84.41 |
| 100 | 2-Phenylacetamide | 103-81-1 | 861.95 | 46.73 | 84.20 |
| 101 | Malonic acid | 141-82-2 | 865.42 | 46.52 | 83.81 |
| 102 | Mitomycin C | 50-07-7 | 867.02 | 46.42 | 83.63 |
| 103 | 2-Deoxyuridine | 951-78-0 | 867.92 | 46.36 | 83.53 |
| 104 | Picrotoxin | 124-87-8 | 868.69 | 46.31 | 83.45 |
| 105 | Muscone | 541-91-3 | 869.43 | 46.27 | 83.36 |
| 106 | Corticosterone | 50-22-6 | 870.00 | 46.23 | 83.30 |
| 107 | Pregnenolone | 145-13-1 | 870.09 | 46.23 | 83.29 |
| 108 | ATP | 56-65-5 | 875.39 | 45.90 | 82.70 |
| 109 | Schisantherin A | 58546-56-8 | 875.42 | 45.90 | 82.70 |
| 110 | DY131 | 95167-41-2 | 878.41 | 45.71 | 82.36 |
| 111 | Oxybutynin chloride | 1508-65-2 | 882.87 | 45.44 | 81.87 |
| 112 | Acitretin | 55079-83-9 | 883.02 | 45.43 | 81.85 |
| 113 | Urolithin A | 1143-70-0 | 884.12 | 45.36 | 81.73 |
| 114 | Veratraldehyde | 120-14-9 | 887.33 | 45.16 | 81.37 |
| 115 | 3-Hydroxyphenylacetic | 621-37-4 | 888.33 | 45.10 | 81.26 |
| | acid | | | | |
| 116 | Adenosine 5- | 61-19-8 | 890.00 | 45.00 | 81.07 |
| | monophosphate | | | | |
| 117 | Juglone | 481-39-0 | 894.51 | 44.72 | 80.57 |
| 118 | Z-Ligustilide | 4431-01-0 | 895.67 | 44.65 | 80.44 |
| 119 | Tropinone | 532-24-1 | 896.55 | 44.59 | 80.34 |
| 120 | Syringaldehyde | 134-96-3 | 896.55 | 44.59 | 80.34 |
| 121 | Gallic acid | 149-91-7 | 898.71 | 44.46 | 80.10 |
| 122 | Creatine monohydrate | 6020-87-7 | 901.68 | 44.28 | 79.77 |
| 123 | DL-Carnitine HCl | 461-05-2 | 902.01 | 44.26 | 79.74 |
| 124 | 5-HTP | 56-69-9 | 902.36 | 44.23 | 79.70 |
| 125 | 1,4-Diaminobutane | 333-93-7 | 902.42 | 44.23 | 79.69 |
| | dihydrochloride | | | | |
| 126 | Diosgenin | 512-04-9 | 903.89 | 44.14 | 79.53 |
| 127 | Ursolic acid | 77-52-1 | 905.10 | 44.06 | 79.39 |
| 128 | Prilocaine | 721-50-6 | 905.23 | 44.06 | 79.38 |
| 129 | Pyrocatechol | 120-80-9 | 906.35 | 43.99 | 79.25 |
| 130 | Isoprenaline HCl | 51-30-9 | 908.26 | 43.87 | 79.04 |
| 131 | Chlorpromazine HCl | 69-09-0 | 908.44 | 43.86 | 79.02 |
| 132 | Homovanillic acid | 306-08-1 | 908.95 | 43.83 | 78.96 |
| 133 | Angelicin | 523-50-2 | 910.28 | 43.74 | 78.82 |
| 134 | Forskolin | 66575-29-9 | 911.32 | 43.68 | 78.70 |
| 135 | Phloretin | 60-82-2 | 912.42 | 43.61 | 78.58 |
| 136 | Cyclocytidine HCl | 10212-25-6 | 914.30 | 43.50 | 78.37 |
| 137 | Reserpine | 50-55-5 | 915.14 | 43.44 | 78.27 |
| 138 | Palonosetron HCl | 135729-62-3 | 915.97 | 43.39 | 78.18 |
| 139 | Capecitabine | 154361-50-9 | 918.89 | 43.21 | 77.86 |
| 140 | Bacitracin Zinc | 1405-89-6 | 919.94 | 43.15 | 77.74 |
| 141 | Ketoisovaleric acid | 759-05-7 | 921.08 | 43.08 | 77.61 |
| 142 | Acipimox | 51037-30-0 | 922.85 | 42.97 | 77.42 |
| 143 | Liensinine | 2586-96-1 | 923.21 | 42.94 | 77.38 |
| 144 | Betamipron | 3440-28-6 | 923.38 | 42.93 | 77.36 |
| 145 | Estradiol valerate | 979-32-8 | 924.65 | 42.86 | 77.22 |
| 146 | 5,15-Diacetyl-3- | 218916-52-0 | 927.14 | 42.70 | 76.94 |
| | benzoyllathyrol | | | | |
| 147 | Uracil | 66-22-8 | 927.30 | 42.69 | 76.92 |
| 148 | Hydrocortisone 21- | 125-04-2 | 927.92 | 42.65 | 76.85 |
| | hemisuccinate (sodium | | | | |
| | salt) | | | | |
| 149 | 4-Methoxyphenylacetic | 104-01-8 | 929.33 | 42.57 | 76.69 |
| | acid | | | | |
| 150 | Cabazitaxel | 183133-96-2 | 929.36 | 42.56 | 76.69 |
| 151 | Ingenol | 30220-46-3 | 930.73 | 42.48 | 76.54 |
| 152 | Vindoline | 2182-14-1 | 930.98 | 42.46 | 76.51 |
| 153 | Sorafenib | 284461-73-0 | 932.25 | 42.39 | 76.37 |
| 154 | 2,2′-Cyclouridine | 3736-77-4 | 935.30 | 42.20 | 76.03 |
| 155 | 4-Pentenoic acid | 591-80-0 | 936.28 | 42.14 | 75.92 |
| 156 | Pyrogallol | 87-66-1 | 936.38 | 42.13 | 75.91 |
| 157 | Cefoselis Sulfate | 122841-12-7 | 937.12 | 42.09 | 75.83 |
| 158 | Scopolamine | 114-49-8 | 937.62 | 42.05 | 75.77 |
| | hydrobromide | | | | |
| 159 | Carvacrol | 499-75-2 | 937.62 | 42.05 | 75.77 |
| 160 | Benzylpenicillin | 113-98-4 | 937.79 | 42.04 | 75.75 |
| | potassium | | | | |
| 161 | Veratric acid | 93-07-2 | 937.88 | 42.04 | 75.74 |
| 162 | D-Pyroglutamic acid | 4042-36-8 | 938.93 | 41.97 | 75.63 |
| 163 | Clarithromycin | 81103-11-9 | 941.49 | 41.82 | 75.34 |
| 164 | Tiglic acid | 80-59-1 | 947.74 | 41.43 | 74.64 |
| 165 | Ginsenoside Rg1 | 22427-39-0 | 947.81 | 41.42 | 74.64 |
| 166 | Quinine | 130-95-0 | 950.73 | 41.24 | 74.31 |
| 167 | Magnolol | 528-43-8 | 950.75 | 41.24 | 74.31 |
| 168 | maleate | 110-16-7 | 951.36 | 41.21 | 74.24 |
| 169 | Spectinomycin | 21736-83-4 | 953.21 | 41.09 | 74.04 |
| | dihydrochloride | | | | |
| 170 | Rhynchophylline | 76-66-4 | 953.80 | 41.05 | 73.97 |
| 171 | Uridine | 58-96-8 | 955.62 | 40.94 | 73.77 |
| 172 | Asperosaponin VI | 39524-08-8 | 955.83 | 40.93 | 73.74 |
| 173 | Lanosterol | 79-63-0 | 957.65 | 40.82 | 73.54 |
| 174 | Hydroquinidine | 1435-55-8 | 958.47 | 40.77 | 73.45 |
| 175 | Cefuroxime axetil | 64544-07-6 | 958.52 | 40.76 | 73.44 |
| 176 | Mianserin HCl | 21535-47-7 | 960.23 | 40.66 | 73.25 |
| 177 | Gefitinib (ZD1839) | 184475-35-2 | 960.31 | 40.65 | 73.25 |
| 178 | Melamine | 108-78-1 | 960.76 | 40.62 | 73.19 |
| 179 | Vitamin C | 50-81-7 | 962.07 | 40.54 | 73.05 |
| 180 | Malic acid | 6915-15-7 | 962.28 | 40.53 | 73.03 |
| 181 | Nordihydroguaiaretic | 500-38-9 | 964.84 | 40.37 | 72.74 |
| | acid | | | | |
| 182 | all-trans Retinal | 116-31-4 | 967.12 | 40.23 | 72.49 |
| 183 | (—)-Borneol | 464-45-9 | 967.20 | 40.23 | 72.48 |
| 184 | 7-Epitaxol | 105454-04-4 | 968.61 | 40.14 | 72.32 |
| 185 | Estradiol Benzoate | 50-50-0 | 970.65 | 40.01 | 72.09 |
| 186 | Tobramycin | 32986-56-4 | 971.48 | 39.96 | 72.00 |
| 187 | Santacruzamate A | 1477949-42-0 | 972.04 | 39.93 | 71.94 |
| | (CAY10683) | | | | |
| 188 | Fraxinellone | 28808-62-0 | 972.50 | 39.90 | 71.89 |
| 189 | N-Acetyl-DL- | 1115-47-5 | 973.29 | 39.85 | 71.80 |
| | methionine | | | | |
| 190 | Apocynin | 498-02-2 | 973.30 | 39.85 | 71.80 |
| 191 | Levothyroxine sodium | 55-03-8 | 974.14 | 39.80 | 71.71 |
| 192 | Guanosine 5′- | 5550-12-9 | 977.65 | 39.58 | 71.31 |
| | monophosphate | | | | |
| | (sodium salt) | | | | |
| 193 | Ginkgolide A | 15291-75-5 | 977.84 | 39.57 | 71.29 |
| 194 | N-Acetylserotonin | 1210-83-9 | 978.08 | 39.55 | 71.27 |
| 195 | Pirarubicin | 72496-41-4 | 978.32 | 39.54 | 71.24 |
| 196 | Acetylvanillin | 881-68-5 | 979.34 | 39.48 | 71.13 |
| 197 | Difloxacin HCl | 91296-86-5 | 980.30 | 39.42 | 71.02 |
| 198 | Calcium D- | 137-08-6 | 981.67 | 39.33 | 70.87 |
| | Panthotenate | | | | |
| 199 | L(+)-2-Aminobutyric | 1492-24-6 | 981.68 | 39.33 | 70.87 |
| | acid | | | | |
| 200 | Artemisinine | 63968-64-9 | 982.40 | 39.29 | 70.78 |
| 201 | Rosavin | 84954-92-7 | 983.74 | 39.20 | 70.64 |
| 202 | Corilagin | 23094-69-1 | 985.42 | 39.10 | 70.45 |
| 203 | DL-Panthenol | 16485-10-2 | 991.50 | 38.72 | 69.77 |
| 204 | Cefotiam hydrochloride | 66309-69-1 | 993.62 | 38.59 | 69.54 |
| 205 | Clenbuterol | 21898-19-1 | 994.53 | 38.54 | 69.44 |
| | (hydrochloride) | | | | |
| 206 | trans-Zeatin | 1637-39-4 | 994.70 | 38.53 | 69.42 |
| 207 | Dopamine HCl | 62-31-7 | 995.96 | 38.45 | 69.28 |
| 208 | Ethisterone | 434-03-7 | 996.32 | 38.43 | 69.24 |
| 209 | Chenodeoxycholic Acid | 474-25-9 | 999.71 | 38.22 | 68.86 |

EPFC fingerprint descriptors and Tanimoto coefficient were used to find additional compounds having >90% structural similarity with the compounds described in Table 2 which stimulate p62-dependent mitophagy in HDFs (Table 3).

TABLE 3

| | | Closest | |
|---|---|---|---|
| # | Compound | Similarities | CAS Number |
| 1 | Hydrolized casein | 0.949 | 65072-00-6 |
| 2 | Totarol | 0.912 | 511-15-9 |
| 3 | Tridecyl salicylate | 0.908 | 19666-16-1 |
| 4 | Honokiol | 0.971 | 35354-74-6 |
| 5 | Oryzanol | 0.917 | 469-36-3 |
| 6 | Thymol | 0.953 | 89-83-8 |
| 7 | Dicapryl Adipate | 0.911 | 31524-62-6 |
| 8 | Myristamidopropyl betaine | 0.909 | 59272-84-3 |
| 9 | Hydroxypinacolone retinoate | 0.910 | 893412-73-2 |
| 10 | Glycyrrhetinic acid | 0.979 | 471-53-4 |
| 11 | Retinyl proprionate | 0.952 | 7069-42-3 |
| 12 | Acetlyphytosphingosine | 0.904 | 21830-28-4 |
| 13 | Isosteric acid | 0.966 | 30399-84-9 |
| 14 | Isopropyl cresols | 0.907 | 3228-02-2 |
| 15 | Abietic acid | 0.924 | 514-10-3 |
| 16 | Oleoyl sarcosine | 0.908 | 110-25-8 |
| 17 | Resveratrol | 0.963 | 501-36-0 |
| 18 | Cocamidopropyl betaine | 0.909 | 86438-79-1 |
| 19 | Sphinganine | 0.928 | 6036-76-6 |
| 20 | Lauryl PCA | 0.936 | 22794-26-9 |
| 21 | Stearyl lactate | 0.907 | 35230-14-9 |
| 22 | Glyceryl myristate | 0.947 | 86195-50-8 |
| 23 | Hydroxystearic acid | 0.952 | 18417-00-0 |
| 24 | Retinyl acetate | 0.972 | 127-47-9 |
| 25 | Oleanolic Acid | 0.984 | 508-02-1 |
| 26 | Glyceryl palmitate | 0.936 | 32899-41-5 |
| 27 | Myristyl lactate | 0.959 | 1323-03-1 |
| 28 | Tannic acid | 0.964 | 18483-17-5 |
| 29 | Retinol | 0.981 | 2052-63-3 |
| 30 | Glyceryl oleate | 0.904 | 67701-32-0 |
| 31 | Sodium ursolate | 0.983 | 220435-39-2 |
| 32 | Quercetin | 0.941 | 117-39-5 |
| 33 | PEG-8 Ricinoleate | 0.907 | 9004-97-1 |
| 34 | Isopropyl lauroyl sarcosinate | 0.902 | 230309-38-3 |
| 35 | Escin | 0.949 | 6805-41-0 |

In one embodiment, the bioactive material may comprise one of the materials listed in Table 2.

In a further embodiment, the bioactive material may comprise one of the materials listed in Table 3.

In a further embodiment, the structure of the bioactive material comprises a phenolic group and derivatives selected from 4-Aminophenol, xanthohumol, fidaxomicin, chloroxine, bisdemethoxycurcumin, oxyresveratrol, 10-gingerol, mycophenolate mofetil, deoxyarbutin, ASC-J9, raspberry ketone, 2,5-dihydroxyacetophenone, sofalcone, capsaicin, hydroxytyrosol, criodictyol, DY131, urolithin A, juglone, syringaldehyde, 5-HTP, pyrocatechol, isoprenaline, homovanillic acid, phloretin, pyrogallol, carvacrol, magnolol, nordihydroguaiaretic acid, apocynin, N-acetylserotonin, corilagin, dopamine, and combinations thereof.

In another embodiment, the structure of the bioactive material comprises at least one of saturated or unsaturated dicarboxylic acids or monocarboxylic acid salts and derivatives selected from malonic acid, itaconic acid, 3-methylglutaric acid, maleic acid, malic acid, fumaric acid, dimethyl fumarate, tiglic acid, 4-pentenoic acid, folinic acid, tartronic acid, 2-aminobutyric acid, chenodeoxycholic acid, deoxycholic acid, carnosic acid, m-hydroxybenzoic acid, folinic acid, mycophenolic acid, 3-hydroxyphenylacetic acid, gallic acid, ursolic acid, 4-methoxyphenylacetic acid, veratric acid, acetylcarnitine, pyroglutamic acid, acipimox, betamipron, and combinations thereof.

In a further embodiment, the structure of the bioactive material comprises at least one primary or secondary amine group salts and derivatives selected from spermine, melamine, 1,4-diaminobutane, and combinations thereof.

In a further embodiment, the structure of the bioactive material comprises at least one keto acid salt or derivative, said acid selected from levulinic acid, ketoisovaleric acid, and combinations thereof.

In a further embodiment, the structure of the bioactive material comprises at least one sugar alcohol selected from ethylene glycol, glycerol, sorbitol, erythritol, threitol, arabitol, ribitol, galactitol, fucitol, iditol, inositol, xylitol, mannitol, maltitol, iso-malt, volemitol, lactitol, maltotriitol, maltotetraitol, polyglycitol, and combinations thereof.

In a further embodiment, the structure of the bioactive material comprises at least one imidazole group or derivative, selected from miconazole, econazole, itraconazole, tioconazole, clotrimazole, and combinations thereof.

In a further embodiment, the bioactive material may comprise at least one of the structures selected from Formula I or Formula II wherein R, R1, R2, and R3 are independently selected from —H; OH; an alkyl selected from a straight-chained, branched or cyclic alkyl; a heterocyclic; heteroalkyl; aryl; heteroaryl; hetero arylalkyl; arylalkyl; tauryl; alkyl ester; and all possible stereoisomers thereof; and wherein R4 is independently selected from H, OH or alkyl. Examples from Formula I include estradiol, estradiol valerate, estriol, or estradiol benzoate. An example from Formula II includes loteprednol etabonate.

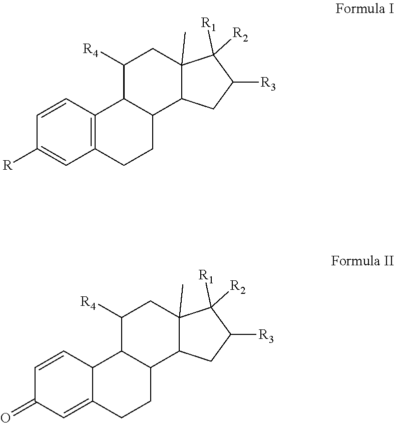

### Example 5. Docking Experiments of Sterane Compound p62-dependent Mitophagy Activators

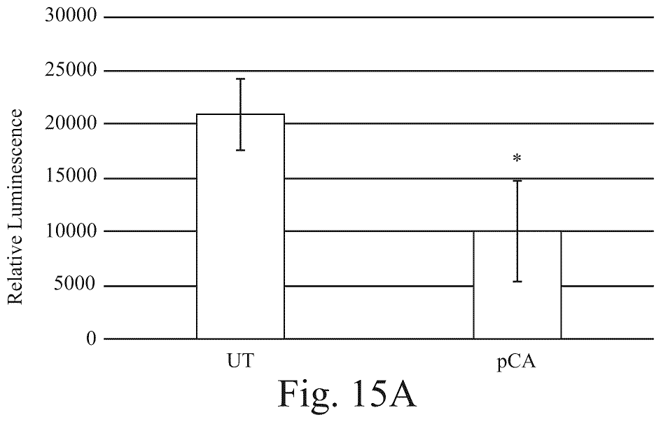

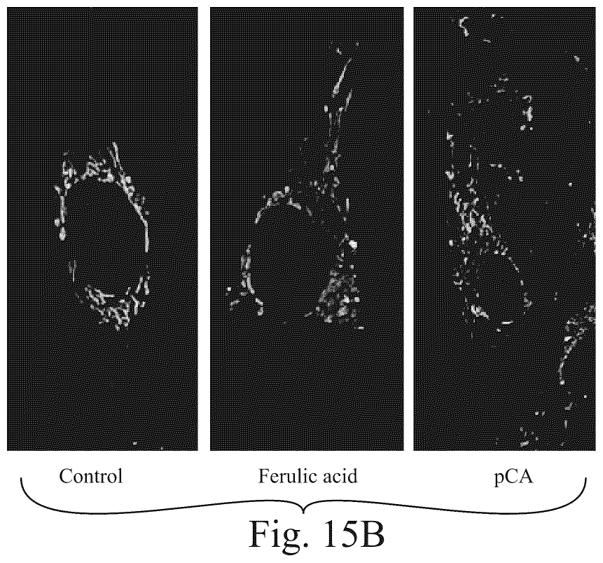

We have demonstrated that one mechanism whereby bioactive materials stimulate p62-dependent mitophagy is through docking to a binding pocket in the ZZ domain of p62, thus promoting receptor oligomerization (Kelly et al.). Oligomerization of p62 has previously been shown to be required for its function as a selective autophagy receptor (SAR) (Wurzer et al.). We further performed in silico docking analysis to assess binding of a subset of p62-depedendent mitophagy activators identified in our compound library screen which contain a common sterane structure to determine their abilities to bind to the ZZ domain of p62 (Method 13). Interestingly, all of these compounds exhibited strong docking scores for p62, supporting the possibility that they may bind directly to this target (FIG. 13). Furthermore, stronger docking predictions correlated with stronger p62-dependent mitophagy (FIG. 13), suggesting a connection between p62 docking efficiency and functional activity of the compounds. Additional sterane structures were evaluated in silico to establish a Markush structure representing p62 ZZ domain-binding sterane compounds (FIGS. 14A-14M).

### Example 6. Hydroxycinnamic Acid Chemistries Stimulate Mitophagy in HDFs and Abrogate Stress-Induced Senescence Phenotypes

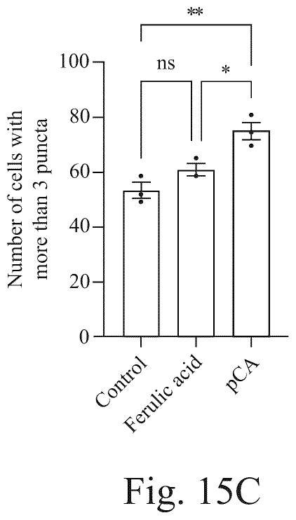

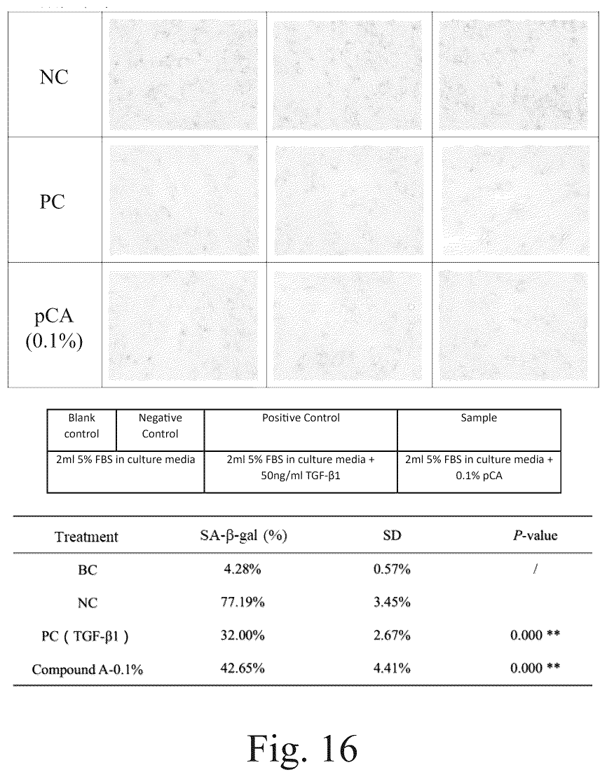

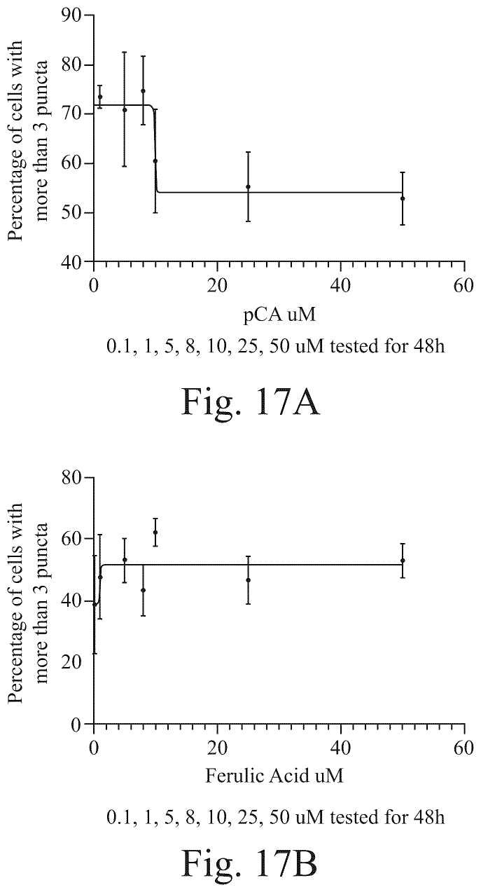

As described herein, we have demonstrated that mitophagy dysfunction is an important mechanism in the development of cellular senescence. Therefore, we further explored the ability of bioactive materials which increase mitophagy levels in skin cells to prevent or rescue cellular senescence and aging phenotypes. Interestingly, hydroxycinnamic acid chemistries (HCAs) were identified as potent mitophagy activators in HDFs (FIGS. 15A-15C) (Methods 3 and 6). Furthermore, HCAs were demonstrated to reduce senescence (FIG. 16) (Method 8). Comparison of different HCA chemistries demonstrated that particular types of HCAs are more effective at increasing basal mitophagy levels in HDFs than others, with a monohydroxycinnamic acid (p-coumaric acid) preferred vs. an O-methylated form (ferulic acid) (FIGS. 17A and 17B). Taken together, HCAs represent a class of novel mitophagy activators which act to combat cellular aging and senescence phenotypes in skin cells exposed to acute and chronic stress.

### REFERENCES

1. Sedlackova, L., and Korolchuk, V. I. (2019). Mitochondrial quality control as a key determinant of cell survival. Biochim. Biophys. Acta Mol. Cell Res. 1866, 575-587. https://doi.org/10.1016/j.bbamcr.2018.12.012.
2. Korolchuk, V. I., Miwa, S., Carroll, B., and von Zglinicki, T. (2017). Mitochondria in cell senescence: is mitophagy the weakest link? EBioMedicine 21, 7-13. https://doi.org/10.1016/j.ebiom.2017.03.020.
3. Gorgoulis, V., Adams, P. D., Alimonti, A., Bennett, D .C., Bischof, O., Bishop, C., Campisi, J., Collado, M., Evangelou, K., Ferbeyre, G., et al. (2019). Cellular senescence: defining a path forward. Cell 179, 813-827. https://doi.org/10.1016/j.cell.2019.10.005.
4. Hill, D.S., Robinson, N. D. P., Caley M. P., Chen M., O'Toole E. A., Armstrong J. L., Przyborski S., Lovat P. E. (2015). Mol. Cancer Ther., 14 (2015), pp. 2665-2673., DOI: 10.1158/1535-7163.MCT-15-0394.
5. Harada, H., Warabi, E., Matsuki, T., Yanagawa, T., Okada, K., Uwayma, J., Ikeda, A., Nakaso, K., Kirii, K., Noguchi, N., Bukawa, H., Siow, R. C. M., Mann, G. E., Shoda, J., Ishii, T. & Sakurai, T. (2013). Deficiency of p62/Sequestosome 1 Causes Hyperphagia Due to Leptin Resistance in the Brain. The Journal of Neuroscience, 33, 14767-14777. DOI: 10.1523/JNEUROSCI.2954-12.2013
6. Brown, A., Patel, S., Ward, C., Lorenz, A., Ortiz, M., DuRoss, A., Wieghardt, F., Esch, A., Otten, E.G., Heiser, L. M., et al. (2016). PEG-lipid micelles enable cholesterol efflux in Niemann-Pick Type Cl disease based lysosomal storage disorder. Sci. Rep. 6, 31750. https://doi.org/10.1038/srep31750.
7. Carroll, B., Otten, E. G., Manni, D., Stefanatos, R., Menzies, F. M., Smith, G. R., Jurk, D., Kenneth, N., Wilkinson, S., Passos, J.F., et al. (2018). Oxidation of SQSTM1/p62 mediates the link between redox state and protein homeostasis. Nat. Commun. 9, 256. https://doi.org/10.1038/s41467-017-02746-z.
8. Zhang, Y., Mun, S. R., Linares, J. F. et al. ZZ-dependent regulation of p62/SQSTM1 in autophagy. Nat Commun 9, 4373 (2018). https://doi.org/10.1038/s41467-018-06878-8.
9. Kelly et al. Suppressed basal mitophagy drives cellular ageing phenotypes which can be reversed by a p62-targeting small molecule.” Developmental Cell (2024): accepted for publication.
10. Mannick, J. B., Lamming, D. W. Targeting the biology of aging with mTOR inhibitors. Nat Aging 3, 642-660 (2023). https://doi.org/10.1038/s43587-023-00416-y.
11. Chung, C. L., Lawrence, I., Hoffman, M. et al. Topical rapamycin reduces markers of senescence and aging in human skin: an exploratory, prospective, randomized trial. GeroScience 41, 861-869 (2019). https://doi.org/10.1007/s11357-019-00113-y.
12. Wurzer, B., Zaffagnini, G., Fracchiolla, D., Turco, E., Abert, C., Romanov, J., and Martens, S. (2015). Oligomerization of p62 allows for selection of ubiquitinated cargo and isolation membrane during selective autophagy. eLife 4, e08941. https://doi.org/10.7554/eLife.08941.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising:
a. bioactive material; and
b. a dermatologically acceptable carrier;
wherein the composition increases p62-dependent mitophagy in human skin cells by at least 30% over baseline as determined by the Luciferase-p62 assay.

2. The composition of claim 1, wherein the structure of the bioactive material comprises a phenolic group and derivatives selected from 4-Aminophenol, xanthohumol, fidaxomicin, chloroxine, bisdemethoxycurcumin, oxyresveratrol, 10-gingerol, mycophenolate mofetil, deoxyarbutin, ASC-J9, raspberry ketone, 2,5-dihydroxyacetophenone, sofalcone, capsaicin, hydroxytyrosol, eriodictyol, DY131, urolithin A, juglone, syringaldehyde, 5-HTP, pyrocatechol, isoprenaline, homovanillic acid, 4-methoxyphenylacetic acid, phloretin, pyrogallol, mecarbinate, carvacrol, magnolol, nordihydroguaiaretic acid, apocynin, N-acetylserotonin, corilagin, dopamine, and combinations thereof.

3. The composition of claim 1, wherein the structure of the bioactive material comprises at least one of saturated or unsaturated dicarboxylic acids or monocarboxylic acid salts and derivatives selected from malonic acid, itaconic acid, 3-methylglutaric acid, malic acid, dimethyl fumarate, tiglic acid, 4-pentenoic acid, folinic acid, L(+) 2-aminobutyric acid, chenodeoxycholic acid, deoxycholic acid, carnosic acid, m-hydroxybenzoic acid, mycophenolic acid, 3-hydroxyphenylacetic acid, gallic acid, ursolic acid, 4-methoxyphenylacetic acid, veratric acid, L-acetylcarnitine hydrochloride, D-pyroglutamic acid, acipimox, betamipron, and combinations thereof.

4. The composition of claim 1, wherein the structure of the bioactive material comprises at least one primary or secondary amine group salts and derivatives selected from spermine, melamine, DL-Panthenol, Clindamycin HCl, Capecitabine, Uridine, 2-Deoxyuridine, 2,2′-Cyclouridine, 1,4-diaminobutane, and combinations thereof.

5. The composition of claim 1, wherein the structure of the bioactive material comprises at least one keto acid salt or derivative, said acid selected from levulinic acid, ketoisovaleric acid, N-Ethylmaleimide, Isovaleroylglycine, 2,3-Butanedione-2-monoxime, Tropinone, Creatine monohydrate, maleate, Vitamin C, N-Acetyl-DL-methionine, DL-Carnitine HCl, and combinations thereof.

6. The composition of claim 1, wherein the structure of the bioactive material comprises at least one imidazole group or derivative, selected from miconazole, miconazole nitrate, econazole nitrate, itraconazole, tioconazole, clotrimazole, Lapatinib Ditosylate, Butenafine HCl, Robenidine hydrochloride, Chlorpromazine HCl, Sorafenib, Mianserin HCl, Gefitinib (ZD1839), Difloxacin HCl, and combinations thereof.

7. The composition of claim 1, wherein the structure of the bioactive material comprises at least one sugar alcohol selected from erythritol, myo-Inositol, inositol, and combinations thereof.

8. The composition of claim 1, wherein the bioactive material comprises one of Formula I, or Formula II in which R, R1, R2, and R3 are independently selected from —H; OH; an alkyl selected from a straight-chained, branched or cyclic alkyl; a heterocyclic; heteroalkyl; aryl; heteroaryl; hetero arylalkyl; arylalkyl; tauryl; alkyl ester; and all possible stereoisomers thereof; and wherein R4 is independently selected from H, OH or alkyl.

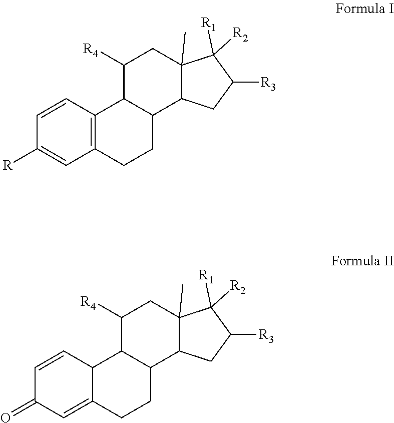

9. The composition of the claim 1, further comprising at least one additional ingredient selected from vitamins, minerals, peptides, sugar amines, sunscreen agents, oil control agents, flavonoids, anti-oxidants, protease inhibitors, tyrosinase inhibitors, anti-inflammatory agents, moisturizing agents, exfoliating agents, skin lightening agents, anti-acne agents, anti-wrinkle agents, phytosterols, N-acyl amino acids, antimicrobials, antifungals, pH adjustors, thickening agents, preservatives, and mixtures thereof.

10. The composition of claim 1, wherein the composition is a skin care composition.

11. The composition of claim 1, wherein the bioactive material is selected from the materials listed in Table 2.

12. The composition of claim 1, further comprising vitamins.

13. A composition comprising:
a. bioactive material selected from the materials in Table 3; and
b. a dermatologically acceptable carrier;
wherein the composition increases p62-dependent mitophagy in human skin cells by at least 30% over baseline as determined by the Luciferase-p62 assay.

14. A skin care composition, comprising:
a. a bioactive material; and
b. a dermatologically acceptable carrier, wherein the composition increases p62-dependent mitophagy in human cells.

15. The composition of claim 14, wherein the composition increases p62-dependent mitophagy in human cells by at least 30% over baseline as determined by the Luciferase-p62 assay.

16. The composition of claim 14, wherein the composition delivers a higher increase in p62-dependent mitophagy in human cells than rapamycin as determined by the Luciferase-p62 assay.

17. The composition of the claim 14, further comprising at least one additional ingredient selected from vitamins, minerals, peptides, sugar amines, sunscreen agents, oil control agents, flavonoids, anti-oxidants, protease inhibitors, tyrosinase inhibitors, anti-inflammatory agents, moisturizing agents, exfoliating agents, skin lightening agents, anti-acne agents, anti-wrinkle agents, phytosterols, N-acyl amino acids, antimicrobials, antifungals, pH adjustors, thickening agents, preservatives, and mixtures thereof.

18. A composition comprising:
a. a hydroxycinnamic acid; and
b. a dermatologically acceptable carrier;
wherein the composition increases mitophagy events in human skin cells and rescues oxidative stress-induced cellular senescence and aging phenotypes.

19. The composition of claim 18, wherein the hydroxycinnamic acid is p-coumaric acid.

20. The composition of the claim 18, further comprising at least one additional ingredient selected from vitamins, minerals, peptides, sugar amines, sunscreen agents, oil control agents, flavonoids, anti-oxidants, protease inhibitors, tyrosinase inhibitors, anti-inflammatory agents, moisturizing agents, exfoliating agents, skin lightening agents, anti-acne agents, anti-wrinkle agents, phytosterols, N-acyl amino acids, antimicrobials, antifungals, pH adjustors, thickening agents, preservatives, and mixtures thereof.

21. The composition of claim 18, wherein the composition is a skin care composition.

22. A method of treating skin, comprising the steps of:
a) identifying a target portion of skin where treatment is desired; and
b) applying a composition to the target portion of skin during a treatment period;
wherein the composition comprises:
a. bioactive material; and
b. a dermatologically acceptable carrier;
wherein the composition increases p62-dependent mitophagy in human cells by at least 30% over baseline as determined by the Luciferase-p62 assay.

23. The method of claim 22, wherein the method improves the occurrence of a visible manifestation and/or functional outcome of skin aging.

24. The method of claim 23, wherein the improvement of skin aging is selected from wrinkles, age spots, fragile skin, barrier integrity, inflammation, hydration, and combinations thereof.

25. The method of claim 22, wherein the method improves the manifestation of a functional outcome of mucosal epithelial aging.

26. The method of claim 25, wherein the improvement of mucosal epithelial aging is selected from vaginal atrophy, gum recession, intestinal permeability, and combinations thereof.

27. The method of claim 22, wherein the method prevents or reduces senescence.

28. The method of claim 22, wherein the method restores skin cells.

29. A method of increasing p62-dependent mitophagy in human cells, the method comprising:
i) identifying a target portion of skin where treatment is desired; and
ii) applying a composition to the target portion of skin during a treatment period;
wherein the composition comprises:
a. bioactive material; and
b. a dermatologically acceptable carrier.

30. The method of claim 29, wherein the composition increases p62-dependent mitophagy in human cells by at least 30% over baseline as determined by the Luciferase-p62 assay.

31. A method of treating skin, comprising the steps of:
a) identifying a target portion of skin where treatment is desired; and
b) applying a composition to the target portion of skin during a treatment period;
wherein the composition comprises:
a. p-coumaric acid; and
b. a dermatologically acceptable carrier;
wherein the composition increases mitophagy events in human skin cells and rescues oxidative stress-induced cellular senescence and aging phenotypes.

32. The method of claim 31, wherein the method improves the occurrence of a visible manifestation and/or functional outcome of skin aging.

33. The method of claim 32, wherein the improvement of skin aging is selected from wrinkles, age spots, fragile skin, barrier integrity, inflammation, hydration, and combinations thereof.

34. The method of claim 32, wherein the method prevents or reduces senescence.

35. The method of claim 32, wherein the method restores skin cells.

36. A method of increasing mitophagy in human cells, the method comprising:
i) identifying a target portion of skin where treatment is desired; and
ii) applying a composition to the target portion of skin during a treatment period;
wherein the composition comprises:
a. p-coumaric acid; and
b. a dermatologically acceptable carrier.
